(19) United States
(12) Patent Application Publication
(10) Pub. No.: US 2007/0004045 A1
(43) Pub. Date: Jan. 4, 2007

(54) Analysis of large numbers of estrogens and other steroids and applications thereof

(76) Inventors: Xia Xu, Frederick, MD (US); Timothy D. Veenstra, Jefferson, MD (US); Larry K. Keefer, Bethesda, MD (US); Regina G. Ziegler, Bethesda, MD (US)

Correspondence Address: WOODCOCK WASHBURN LLP, ONE LIBERTY PLACE - 46TH FLOOR, PHILADELPHIA, PA, 19103, US

(21) Appl. No.: 11/449,484

(22) Filed: Jun. 7, 2006

Related U.S. Application Data
(60) Provisional application No. 60/756,768, filed on Jan. 6, 2006.
(60) Provisional application No. 60/688,160, filed on Jun. 7, 2005.

(51) Int. Cl. G01N 33/00

(52) U.S. Cl. 436/87

(57) ABSTRACT

Mass-spectrometry based methods of analyzing estrogens and other steroids from biological samples are disclosed herein. Further disclosed are methods of detecting the presence of illegal steroids in a mammal. Methods of detecting a disease state or condition or elevated risk of a disease state or condition in a mammal are also disclosed. Also disclosed are kits for use in a method for detecting and/or quantifying one or more steroids in a sample by mass spectrometry.

### CROSS-REFERENCE TO RELATED APPLICATIONS

This application claims the benefit of U.S. provisional application Ser. Nos. 60/688,160 filed Jun. 7, 2005 and 60/756,768 filed Jan. 6, 2006. The entirety of each of these patent applications is incorporated by reference herein.

### STATEMENT OF GOVERNMENT SUPPORT

The work leading to the disclosed inventions was funded in whole or in part with Federal funds from the National Cancer Institute, National Institutes of Health, under Contract No. NO1-CO-12400. Accordingly, the U.S. Government has rights in these inventions.

### FIELD OF THE INVENTION

The disclosed invention is generally related to the field of analytical chemistry. More specifically, the disclosed invention is related to the field of analyzing biologic samples for the presence of steroids and metabolites thereof. The disclosed invention also is related to the fields of disease detection, prevention, and treatment.

### BACKGROUND OF THE INVENTION

The evidence that endogenous estrogens play a role in the development of breast cancer is substantial. Increased breast cancer risk has been reported in women with high circulating and urinary estrogen levels, as well as in those exposed to increased estrogens over time as a result of early onset of menstruation, late menopause, postmenopausal obesity, and/or postmenopausal use of replacement hormones. Although the exact mechanism is not fully elucidated, there are two leading hypotheses regarding the role of estrogens in breast carcinogenesis. One of these hypotheses involves catechol estrogens, mainly 2-hydroxyestrone, 2-hydroxyestradiol, and 4-hydroxyestrone (FIG. 1), reacting with DNA to form both stable and depurinating adducts and causing other types of oxidative DNA damage that can lead to cell transformation and cancer initiation. Alternatively, it has been proposed that the potent mitogenic effects of estrogen are key mechanisms leading to carcinogenesis. In this hypothesis, the 16α-hydroxylated estrogens, such as 16α-hydroxyestrone, would be responsible for breast carcinogenesis due to their much stronger hormonal and mitogenic activity compared to the catechol estrogens.

Current methods for measuring endogenous estrogen metabolites have involved radioimmunoassay (RIA), enzyme immunoassay (EIA), high-performance liquid chromatography (HPLC) with electrochemical detection, and stable isotope dilution combined with analysis using gas chromatography-mass spectrometry (GC-MS). Although RIA and EIA can be sensitive, they often suffer from poor specificity, accuracy, and/or reproducibility due to the cross-reactivity and lot-to-lot variation of antibodies. Although HPLC with electrochemical detection has been used for estrogen metabolite analysis in hamsters treated with 17β-estradiol and in pregnant women whose estrogen levels are elevated at least 10-fold, it is relatively insensitive. Its specificity and accuracy for measuring endogenous levels of estrogen metabolites in human biological matrices are questionable. In contrast, the stable isotope dilution GC-MS method is sensitive, specific, and accurate, and has been successfully used for urine samples from both non-pregnant premenopausal women and postmenopausal women, in which the absolute levels of endogenous estrogen metabolites are substantially reduced. Unfortunately, this method is extremely laborious, requiring many steps of solid phase extractions, ion-exchange column separations, and liquid-liquid extractions, as well as two chemical derivatization procedures for each urine sample.

In an attempt to improve the efficiency of the extraction and analysis of estrogens and other steroids from biological samples, methods using HPLC-electrospray ionization (ESI)-MS and ESI-MSⁿ for measuring endogenous ketolic estrogens and estrogen metabolites in pre- and postmenopausal urine have been reported in International PCT patent application WO03/089921A1, “Methods for Separation and Detection of Ketosteroids and Other Carbonyl-Containing Compounds” by Xu, et al. Although these methods overcome many of the earlier problems, the analytes are derivatized with an ionizable moiety to facilitate MS detection; and steroid degradation during derivatization remains a problem. Xu et al. solves this problem by providing a two-step derivatization procedure in which the carbonyl groups on certain estrogens and estrogen metabolites (i.e., the ketolic estrogens) are first protected, followed by derivatization of one or more hydroxyl groups in the second step. Although Xu, et al. disclose that this two-step process provides better HPLC separation of steroids, and allows for better signal detection in API-MS (such as ESI-MS), higher efficiency processes are greatly desired in order to bring these techniques into the clinic.

Accordingly, there is currently a need for efficient processes for analyzing both ketolic and non-ketolic estrogens and other steroids in biological samples. For example, reducing the number of derivatization steps for attaching ionizable moieties to estrogens from two to one is presently needed. Indeed, reducing the number of derivatization steps needed for ESI-MSⁿ analysis also reduces the amount of biological sample, e.g., urine or blood, needed for analysis. There is presently a need for methods to quantify a large number of ketolic and non-ketolic estrogens and other steroids from small quantities of urine.

### SUMMARY OF THE INVENTION

The methods provided herein are capable of accurately and precisely measuring the absolute quantities of many e.g., fifteen or more ketolic and non-ketolic estrogens and their metabolites, including keto, methoxylated, and hydroxylated metabolites, found in urines and other biologic samples from pre- and postmenopausal women, as well as men.

The present invention provides methods of analyzing the quantitative levels of individual estrogens in a biological sample, such as urine or blood, the methods comprising: extracting estrogens from a sample to provide a concentrated sample, the estrogens comprising an estrogen, an estrogen metabolite, or any combination thereof, reacting estrogens in the concentrated sample in a single derivatization step with a hydroxyl protecting reagent under pH conditions between about 7 and about 11.5 in the presence of a reducing agent, an anti-oxidant, or both, to form estrogen derivatives, the concentrated sample optionally comprising at least one ketolic steroid or metabolite thereof, the estrogen derivatives comprising one or more derivatives of one or more estrogens, one or more derivatives of one or more estrogen metabolites, or any combination thereof, at least partially purifying the estrogen derivatives, such as by using chromatography; and analyzing the purified estrogen derivatives by mass spectrometry to ascertain the amount of each estrogen or estrogen metabolite in the sample.

The present invention also provides methods of detecting a disease state or condition, or elevated risk of a disease or condition, in a mammal, comprising: obtaining a biologic sample from the mammal, such as urine or blood; extracting steroids from the sample to provide a concentrated sample, the steroids comprising a steroid, a steroid metabolite, or any combination thereof, reacting steroids in the concentrated sample in a single derivatization step with a hydroxyl protecting reagent under pH conditions between about 7 and about 11.5 in the presence of a reducing agent, an anti-oxidant, or both, to form steroid derivatives, the concentrated sample optionally comprising at least one ketolic steroid or metabolite thereof, the steroid derivatives comprising one or more derivatives of one or more steroids, one or more derivatives of one or more steroid metabolites, or any combination thereof; at least partially purifying the steroid derivatives; analyzing the purified steroid derivatives by chromatography/mass spectrometry to provide a steroid/steroid metabolite profile of the sample; and comparing the steroid/steroid metabolite profile of the sample to steroid/steroid metabolite profiles indicative of a disease state or condition, elevated risk of a disease state or condition, or absence of a disease state or condition to ascertain the presence or absence of the disease or condition in the mammal or the likelihood of contracting the disease or having the condition by the mammal.

The present invention further provides methods of testing a mammal for the presence of illegal steroids comprising: obtaining a biologic sample from the mammal, such as urine or blood; extracting steroids from the sample to provide a concentrated sample, the steroids comprising a steroid, a steroid metabolite, or any combination thereof; reacting steroids in the concentrated sample in a single derivatization step with a hydroxyl protecting reagent under pH conditions between about 7 and about 11.5 in the presence of a reducing agent, an anti-oxidant, or both, to form steroid derivatives, the concentrated sample optionally comprising at least one ketolic steroid or metabolite thereof, the steroid derivatives comprising one or more derivatives of one or more steroids, one or more derivatives of one or more steroid metabolites, or any combination thereof; at least partially purifying the steroid derivatives; analyzing the purified steroid derivatives by mass spectrometry to ascertain the amount of various steroids in the sample; and comparing the amount of steroids in the sample with a threshold amount as determined by applicable federal, state, local, association or league rules.

The present invention also provides kits for use in a method for detecting one or more steroids in a sample by chromatography/mass spectrometry, the kits comprising in packaged combination: an antioxidant, reducing agent, or any combination thereof; a standard of one or more deuterated steroids; a hydroxyl protecting reagent; and a derivatization buffer characterized as having a pH in the range of from about 7 to about 11.5.

Other aspects of the present invention will be apparent to those skilled in the art in view of the detailed description and drawings of the invention as provided herein.

### BRIEF DESCRIPTION OF THE DRAWINGS

The foregoing summary, as well as the following detailed description, is further understood when read in conjunction with the appended drawings. For the purpose of illustrating the invention, there is shown in the drawings exemplary embodiments of the invention; however, the invention is not limited to the specific methods, compositions, and devices disclosed. In the drawings:

FIG. 1 depicts estrogens and endogenous estrogen metabolism in humans;

FIG. 2 is a schematic illustration of an embodiment of a method of the present invention for the analysis of approximately fifteen endogenous estrogens and their metabolites from biologic specimens;

FIG. 3 provides a graphical representation of protection of catechol estrogens by L-ascorbic acid during dansyl derivatization especially when subject to high pH; the same amounts of catechol estrogens were subject to dansylation at increasing pH without L-ascorbic acid (A) or with L-ascorbic acid (B);

FIG. 4 depicts examples of mass spectra showing the measurement of the dansylated derivatives of 17β-estradiol (A) and 2-hydroxyestradiol (B);

FIG. 5 depicts high performance liquid chromatography-electrospray ionization-tandem mass spectrometry selected reaction monitoring (SRM) chromatographic profiles of dansylated derivates of estrogen and its metabolites corresponding to (A) a 0.12-ng EM/mL urine quality control sample, (B) a 0.5 mL pooled premenopausal urine sample, (C) a 0.5 mL pooled postmenopausal urine sample, and (D) a 0.5 mL pooled male urine sample. (i) E₁, (ii) E₂, (iii) 16-ketoE₂ and 16α-OHE₁, (iv) E₃, 16-epiE₃, and 17-epiE₃, (v) 3-MeOE₁, 2-MeOE₁, and 4-MeOE₁, (vi) 2-MeOE₂ and 4-MeOE₂, (vii) 2-OHE₁, and 4-OHE₁, and (viii) 2-OHE₂;

FIG. 6 (A-H) depict mass spectrometry peak area ratio versus concentration calibration curves for the detection of various estrogens;

FIG. 7 depicts urinary endogenous EM excretion in postmenopausal women, premenopausal women, and men (data expressed as mean and standard error);

FIGS. 8A and 8B depict flow charts for methods of measuring serum unconjugated (A) and unconjugated+conjugated (B) estrogens;

FIG. 9 depicts HPLC-ESI-MS² results for a serum calibration sample;

FIG. 10 depicts HPLC-ESI-MS² results using serum of premenopausal luteal phase in women (A—unconjugated estrogens; B—unconjugated+conjugated estrogens);

FIG. 11 depicts HPLC-ESI-MS² results using serum of premenopausal follicular phase in women (A—unconjugated estrogens; B—unconjugated+conjugated estrogens); and

FIG. 12 depicts HPLC-ESI-MS² results using serum of postmenopausal women (A—unconjugated estrogens; B—unconjugated+conjugated estrogens).

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

As employed above and throughout the disclosure, the following terms, unless otherwise indicated, shall be understood to have the following meanings.

Alkaline conditions: Having a pH greater than 7.

API: Atmospheric pressure ionization. This term includes (without limitation) both ESI and APCI.

APCI: atmospheric pressure chemical ionization, which is another example of an ionization method that can be used in ionization mass spectroscopy.

Catechol estrogens (CE): estrogens having an aromatic ring bearing two hydroxyl groups.

dCE: deuterated analogs of catechol estrogens.

Carbonyl-bearing compound: a compound having as part of its structure a carbon-oxygen double bond.

Chromatographic separation: A separation method that depends upon the different rates at which various substances moving in a stream (mobile phase) are retarded by a stationary material (stationary phase) as they pass over it. In liquid chromatography, the mobile phase is liquid. Higher performance liquid chromatography (“HPLC”) refers to systems which obtain excellent resolution by forcing the mobile phase under pressure through a long, usually thin column. Examples of HPLC pressures are 350-1500 psi, although the pressure may be higher (for example as high as 10,000 psi). Gas chromatography (“GC”) can also be suitably used in various embodiments of the invention for separation.

Detecting: a qualitative measurement, a quantitative measurement, or both, of a compound in a sample.

ESI-MS: electrospray ionization mass spectrometry, which is a particular example of ionization spectroscopy.

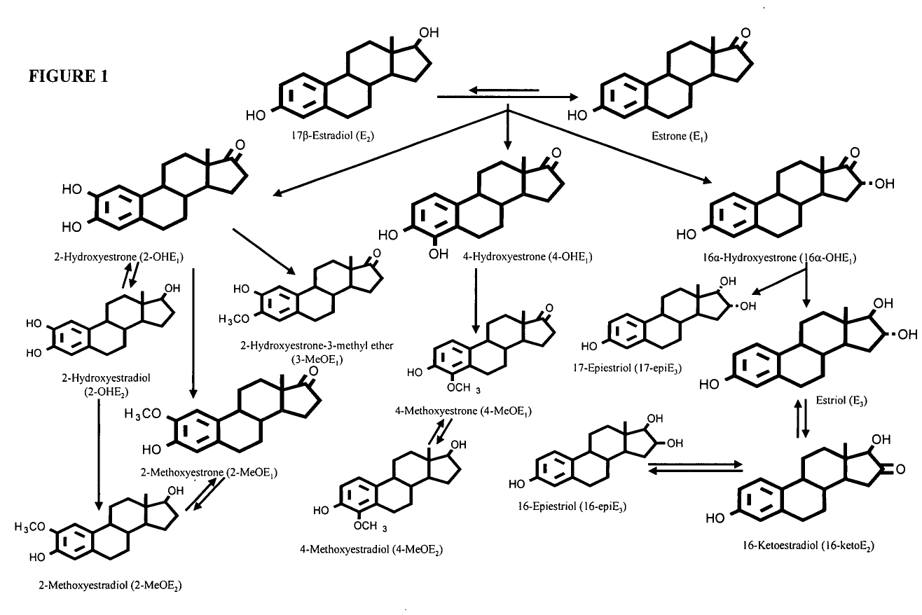

Estrogens: one or more estrogens, an estrogen metabolite, or any combination thereof, examples of which are included in FIG. 1.

HPLC: high performance liquid chromatography, a liquid chromatographic method of separation that includes the techniques of nano-LC and capillary HPLC.

HPLC-ESI-MS: high performance liquid chromatography-electrospray ionization-mass spectrometry, a specific type of LC-MS in which ESI is the ionization method.

Ionization spectroscopy: Spectroscopy preceded by ionization of the analyte, for example by gas-phase ionization, electron ionization, chemical ionization (such as desorption chemical ionization), negative ion chemical ionization, and atmospheric pressure ionization (such as electrospray ionization and atmospheric chemical ionization).

Ketosteroid: a carbonyl-bearing steroid.

Non-ketosteroid: a steroid not bearing a carbonyl group

LC-MS: liquid chromatography-mass spectrometry.

SIM: single ion monitoring.

Steroids: one or more steroid hormones or metabolites thereof. The class of steroids includes progestagens, glucocorticoids, mineralocorticoids, androgens, and estrogens.

In various embodiments, methods of analyzing the presence of estrogens in a biological sample are provided. These methods typically comprise the steps of extracting estrogens from a biological sample (e.g., blood, plasma, serum, saliva, lymph fluid, cellular interstitial fluid, mucus, spinal fluid, tissue, breast nipple aspirate, breast duct lavage, and especially urine) and reacting the extracted estrogens with a suitable hydroxyl protecting reagent in the presence of a suitable reducing agent, an anti-oxidant, or both. The hydroxyl protecting reagent is suitably selected so that it contains an ionizable group and enables the estrogen derivatives to be detected using mass spectrometry. Prior to MS detection, the estrogen derivatives are suitably at least partially purified and separated, such as by use of liquid chromatography.

Suitable estrogens or one or more metabolites thereof can be of endogenous or exogenous origin. Endogenous estrogens are typically naturally synthesized within a mammal undergoing ordinary respiration and metabolism. Exogenous estrogens on the other hand are typically synthetically derived, or derived from other natural sources such as animals (e.g., cows) and enter a mammal artificially, such as by injection, inhalation, absorption, and the like. Any of the known estrogens or one or more metabolites thereof that are found in mammals, especially humans, can be analyzed according to the methods provided herein.

In one embodiment, a method of analyzing the presence of estrogens in a biological sample first includes one or more steps to extract estrogens from a biological sample, such as urine, to provide a concentrated sample, the estrogens comprising an estrogen, an estrogen metabolite, or any combination thereof. Since endogenous estrogens and their metabolites in urine are mostly present as glucuronide conjugates and small amounts of sulfate conjugates, a hydrolysis step can be suitably included to deconjugate them prior to extraction. Accordingly, the extracting step used in various embodiments suitably includes hydrolyzing conjugates of estrogens present in the biological samples. For example, to a urine sample is added an enzymatic hydrolysis buffer containing β-glucuronidase/sulfatase from Helix pomatia (Type H-2) and a suitable buffer (pH 4.1). The sample is incubated, typically overnight at 37° C., to hydrolyze and deconjugate the estrogens.

In embodiments where the estrogens are hydrolyzed, the estrogens can be subsequently extracted from the aqueous phase using a suitable organic solvent, such as dichloromethane. In one embodiment it is particularly preferred that the hydrolyzed estrogens are subjected to slow inverse extraction Slow inverse extraction is suitably achieved using an organic solvent for at least about 10 minutes, preferably for at least about 30 minutes. After extraction, the aqueous layer can be discarded and the organic solvent portion can be evaporated to dryness.

The extracted estrogens are then reacted in the concentrated sample in a single derivatization step with a hydroxyl protecting reagent under basic pH conditions in the presence of a reducing agent, an anti-oxidant, or both, to form estrogen derivatives.

Suitable hydroxyl protecting reagents comprise a compound that forms a silyl derivative, an acyl derivative, a benzoyl derivative, an alkyl derivative, a dansyl derivative, a nitrobenzofuran derivative, or any combination thereof. Specific hydroxyl protecting reagents within these classes include nitrobenzopentafluorobenzoyl hydroxylamine, hydroxylamine, dabsyl chloride, dansyl chloride, 1-fluoro-2,4-dinitrobenzene, 4-fluoro-3-nitrobenzofurazan, or any combination thereof. Among these, dansyl chloride is a particularly preferred hydroxyl protecting reagent.

The derivatization of the estrogens with the hydroxyl protecting group is suitably carried out under basic conditions, suitably at a pH of between about 7 and about 11.5, and more suitably with a pH in the range of from about 8.5 to 10.5. The estrogens are suitably reacted at a temperature less than about 100° C., typically less than 80° C., typically at least about 35° C., or at least about 50° C. for a time suitable to ensure protection of the hydroxyl groups present on the estrogens. Suitable reaction times at these conditions are typically less than about 20 minutes, more typically less than about 10 minutes, and even more typically less than about five minutes. In certain embodiments, the derivatization step is typically completed prior to purifying the estrogen derivatives.

The anti-oxidant, reducing agent, or both, can be added anywhere along the method as long as it is present during the reacting step wherein the estrogens are derivatized using the suitable hydroxyl protecting agent. In certain embodiments, the anti-oxidant, reducing agent, or both is conveniently added to the sample prior to the extraction step. Suitable anti-oxidants that can be used in various embodiments include an ascorbic acid, a tocopherol, a carotenoid, beta carotene, butylated hydroxyanisole, butylated hydroxytoluene, propyl gallate, trihydroxybutyrophenone, uric acid, or any combination thereof. Many food preservatives known to be effective in the food, nutritional, and pharmaceutical industry may also be suitably used as an anti-oxidant. A particularly suitable anti-oxidant includes L-ascorbic acid. The concentration of the anti-oxidant, reducing agent, or both, is suitably in the range of from about 0.01 percent (w/v) to about 1 percent (w/v) based on volume of the concentrated sample. In certain embodiments, the concentrated sample is substantially composed of estrogens and the concentration of the anti-oxidant is suitably in the range of from about 0.01 percent (w/v) to about 1 percent (w/v) based on volume of the estrogens.

In certain embodiments, the concentrated sample may include at least one ketolic estrogen or metabolite thereof, the estrogen derivatives comprising one or more derivatives of one or more estrogens, one or more derivatives of one or more estrogen metabolites, or any combination thereof. The hydroxyl protecting agent suitably reacts only with hydroxyl groups on the estrogens. The ketolic estrogens present in the sample do not require first protecting the carbonyl group with a protecting agent.

After the estrogens are derivatize with the hydroxyl protecting group, the estrogen derivatives are at least partially purified using a suitable procedure that separates and purifies them according to differences in their molecular characterization (e.g., molecular mass, polarity, hydrophobicity, and the like). As used herein, the phrase “at least partially” means partially, essentially completely, or completely. Accordingly, partially purified estrogens may contain some non-estrogen components, for example, water, solvents, salts, molecular fragments, contaminants, and the like. Essentially completely purified estrogens contain essentially no non-estrogen components; non-estrogen components may be present to the extent that there is no detectable difference in properties between essentially completely purified estrogens and completely purified estrogens. A suitable separation/purification procedure includes a chromatographic procedure, for example liquid chromatography (LC). HPLC is preferably used.

Purification and separation of the estrogen derivatives is suitably carried out using a liquid chromatography method, such as HPLC. In certain embodiments, the estrogens can be reacted with a hydroxyl protecting reagent prior to, simultaneously with, or subsequent to separation by liquid or gas chromatography. Reverse-phase HPLC can also be used in certain embodiments, which is preferably performed using a gradient solution comprising a suitable alcohol and an aqueous acidic solution. A suitable alcohol is methanol and a suitable aqueous acidic solution is aqueous formic acid. A non-polar stationary phase is also suitably used, examples of which includes a C18 stationary phase. Suitable gradients include gradient elutions from about 70:30 to about 85:15, based on volume, of methanol:aqueous formic acid solution.

In certain embodiments of the present invention, a method is provided that further includes the step of adding a standard of one or more estrogens to the sample, to the concentrated sample, into the purified sample prior to MS detection, or any combination thereof. The standards may be non-deuterated, but are preferably deuterated for conducting quantitative analysis on the mass spectra. Suitable deuterated standards of estrogens and other steroids and metabolites thereof are commercially available, such as from C/D/N Isotopes, Inc. (Pointe-Claire, Quebec, Canada), Medical Isotopes, Inc. (Pelham, N.H.). Non-deuterated standards are available from Alltech Applied Science Labs (State College, Pa., www.alltechWEB.com). Various embodiments include one, two, three, four, five, six, seven, eight, nine, ten, 11, 12, 13, 14, 15, or even 16 or more standards. Combinations or deuterated and non-deuterated standards are envisioned. Greater numbers of deuterated standards (e.g., five or more) used in certain embodiments of the methods of the present invention affords the ability to quantify a large number (e.g., eight, nine, 10, 11, 12, 13, 14, 15 or even 16 or more) of estrogens. Quantitation of estrogens in urine, for example, can be carried out using commercially available software analysis tools for use with mass spectrometers, an example of which includes Xcalibur™ Quan Browser, available from ThermoFinnigan (Thermo Electron Corporation, Waltham, Mass.). Calibration curves for a number of estrogens and other steroids and metabolites thereof can be constructed by plotting peak area ratios of the non-deuterated sample peak to the deuterated peak obtained from calibration standards versus amounts of the deuterated standards and fitting these data using linear regression with 1/X weighting. The amount of estrogens and other steroids in samples can then be interpolated using this linear function. The deuterium labeled standards without exchange loss are preferably used. Suitable deuterated estrogen standards include the following: d₄-E₂ can be used as the internal standard for E₂ and E₁; d₃-E₃ for E₃, 16-KetoE₂, and 16α-OHE₁; d₃-16-epiE₃ for 16-epiE₃ and 17-epiE₃; d₅-2-MeOE₂ for 2-MeOE₂, 4-MeOE₂, 2-MeOE₁, 4-MeOE₁, and 3-MeOE₁; d₅-2-OHE₂ for 2-OHE₂, 2-OHE₁, and 4-OHE₁. In preferred embodiments, all five of these deuterated estrogen standards are used.

After the estrogen derivatives are suitably purified, they are analyzed using mass spectrometry (MS), along with any deuterated standards. Atmospheric pressure ionization mass spectrometry, such as positive ion mode electrospray mass spectrometry, is preferably used. It is further preferred to use HPLC-ESI-MSⁿ to ascertain the amount of estrogens in the samples. Procedures for analyzing estrogens using MS are generally provided in U.S. Patent Appl. No. 2005/0181514, “Methods for Separation and Detection of Ketosteroids and Other Carbonyl-Containing Compounds”, by Xu, et al., the portion of which pertaining to HPLC-ESI-MS is incorporated by reference herein.

The aforementioned methods and procedures can be extended to detecting a disease state or condition in a mammal. Accordingly, in various embodiments the present invention also provides methods of detecting a disease state or condition in a mammal. In these embodiments, the methods include obtaining a suitable sample from the mammal, such as urine, and extracting steroids from the sample to provide a concentrated sample. The steroids in the concentrated sample are then reacted in a single derivatization step using a hydroxyl protecting reagent under basic pH conditions in the presence of a reducing agent, an anti-oxidant, or both, to form steroid derivatives, the concentrated sample optionally comprising at least one ketolic steroid or metabolite thereof, the steroid derivatives comprising one or more derivatives of one or more steroids, one or more derivatives of one or more steroids metabolites, or any combination thereof. The steroid derivatives are at least partially purified using a suitable purification methodology, such as liquid chromatography. The purified steroid derivatives are then analyzed by mass spectrometry to provide a steroid metabolite profile of the sample, and the steroid metabolite profile of the sample is compared to a steroid metabolite profile indicative of a disease state or condition to ascertain the presence of the disease or condition in the mammal or the likelihood of contracting the disease or having the condition by the mammal. In these embodiments, the steroid or one or more metabolites thereof is suitably an estrogen or one or more metabolites thereof. Accordingly, the disease state or condition suitably comprises breast cancer, other hormone-related cancers, an endocrine disease, infertility, or any combination thereof.

The aforementioned methods described hereinabove can also be adapted for methods of testing a mammal for the presence of illegal steroids. Accordingly, in several embodiments there are provided methods of testing a mammal for the presence of illegal steroids comprising the steps of: obtaining a sample from a mammal, such as urine; extracting steroids from a sample to provide a concentrated sample, the steroids comprising a steroid, a steroid metabolite, or any combination thereof; reacting steroids in the concentrated sample in a single derivatization step with a hydroxyl protecting reagent under basic pH conditions in the presence of a reducing agent, an anti-oxidant, or both, to form steroid derivatives, the concentrated sample optionally comprising at least one ketolic steroid or metabolite thereof, the steroid derivatives comprising one or more derivatives of one or more steroids, one or more derivatives of one or more steroids metabolites, or any combination thereof; at least partially purifying the steroid derivatives by liquid chromatography; analyzing the purified steroid derivatives by mass spectrometry to ascertain the amount of steroids in the sample; and comparing the amount of steroids in the sample with a threshold amount as determined by applicable federal, state, local, association or league rules.

Various embodiments of the present invention for methods of testing for the presence of illegal steroids can be applied essentially to any animal, preferably a mammal. Examples of suitable mammals that these methods can be applied to include humans, horses, sheep, goats, cats, dogs, pigs, guinea pigs, monkeys, cows, rats, and mice. Among humans, it is particularly useful to use these method for screening athletes for the presence of illegal steroids, precursors of illegal steroids, metabolites of illegal steroids, metabolites of precursors of illegal steroids, and other performance-enhancing drugs and the like. The methods provided herein can be suitably used by any of the known sporting associations for screening its athletes. Examples of sporting associations that an athlete may be a participant of include one or more of the International Olympic Committee (IOC), the National Basketball Association (NBA), the National Hockey League (NHL), the National Football League (NFL), the Major League Baseball (MLB), the National Collegiate Athletic Association (NCAA), the Association of Tennis Players (ATP), the World Boxing Organization (WBO), World Boxing Association (WBA), World Boxing Council (WBC), and the like. When comparing the amount of steroids in the sample with a threshold amount as determined by applicable federal, state, local, association or league rules, the threshold amount is suitably the detectable limit of an illegal steroid.

Suitable steroids or one or more metabolites thereof can be of endogenous or exogenous origin. Endogenous steroids are typically naturally synthesized within a mammal undergoing ordinary respiration and metabolism. Exogenous steroids on the other hand are typically synthetically derived and enter a mammal artificially, such as by injection, inhalation, injection, absorption, and the like. Any of the known steroids or one or more metabolites thereof that are found in mammals, especially humans, can be analyzed according to the methods provided herein. In certain embodiments, such as in correlating certain steroid metabolites with cancer, suitable steroids include the catechol steroids, or one or more metabolites thereof. Examples of the catechol steroids that can be detected according to the methods of the present invention include 2-hydroxyestrone, 2-hydroxyestradiol, 4-hydroxyestrone, and the like.

In one embodiment, a method of analyzing the presence of steroids in a biological sample first includes one or more steps to extract steroids from a biological sample, such as urine, to provide a concentrated sample, the steroids comprising an steroid, an steroid metabolite, or any combination thereof. Since endogenous steroids and their metabolites in urine are mostly present as glucuronide conjugates and small amounts of sulfate conjugates, a hydrolysis step can be suitably included to deconjugate them prior to extraction. Accordingly, the extracting step used in various embodiments suitably includes hydrolyzing conjugates of steroids present in the biological samples. For example, to a urine sample is added an enzymatic hydrolysis buffer containing β-glucuronidase/sulfatase from Helix pomatia (Type H-2) and a suitable buffer (pH 4.1). The sample is incubated, typically overnight at 37° C., to hydrolyze and deconjugate the steroids.

In embodiments where the steroids are hydrolyzed, the steroids can be subsequently extracted from the aqueous phase using a suitable organic solvent, such as dichloromethane. In one embodiment it is particularly preferred that the hydrolyzed steroids are subjected to slow inverse extraction Slow inverse extraction is suitably achieved using an organic solvent for at least about 10 minutes, preferably for at least about 30 minutes. After extraction, the aqueous layer can be discarded and the organic solvent portion can be evaporated to dryness.

The extracted steroids are then reacted in the concentrated sample in a single derivatization step with a hydroxyl protecting reagent under basic pH conditions in the presence of a reducing agent, an anti-oxidant, or both, to form steroid derivatives.

Suitable hydroxyl protecting reagents comprise a compound that forms a silyl derivative, an acyl derivative, a benzoyl derivative, an alkyl derivative, a dansyl derivative, a nitrobenzofuran derivative, or any combination thereof. Specific hydroxyl protecting reagents within these classes include nitrobenzopentafluorobenzoyl hydroxylamine, hydroxylamine, dabsyl chloride, dansyl chloride, 1-fluoro-2,4-dinitrobenzene, 4-fluoro-3-nitrobenzofurazan, or any combination thereof. Among these, dansyl chloride is a particularly preferred hydroxyl protecting reagent.

The derivatization of the steroids with the hydroxyl protecting group is suitably carried out under basic conditions, suitably at a pH of between about 7 and about 11.5, and more suitably with a pH in the range of from about 8.5 to 10.5. The steroids are suitably reacted at a temperature less than about 100° C., typically less than 80° C., typically at least about 35° C., or at least about 50° C. for a time suitable to affect deprotection of the hydroxyl groups present on the steroids. Suitable reaction times at these conditions are typically less than about 20 minutes, more typically less than about 10 minutes, and even more typically less than about five minutes. In certain embodiments, the derivatization step is typically completed prior to purifying the steroid derivatives.

The anti-oxidant, reducing agent, or both, can be added anywhere along the method as long as it is present during the reacting step wherein the steroids are derivatized using the suitable hydroxyl protecting agent. In certain embodiments, the anti-oxidant, reducing agent, or both is conveniently added to the sample prior to the extraction step. Suitable anti-oxidants that can be used in various embodiments include an ascorbic acid, a tocopherol, a carotenoid, beta carotene, butylated hydroxyanisole, butylated hydroxytoluene, propyl gallate, trihydroxybutyrophenone, uric acid, or any combination thereof. Many food preservatives known to be effective in the food, nutritional, and pharmaceutical industry may also be suitably used as an anti-oxidant. A particularly suitable anti-oxidant includes L-ascorbic acid. The concentration of the anti-oxidant, reducing agent, or both, is suitably in the range of from about 0.01 percent (w/v) to about 1 percent (w/v) based on volume of the concentrated sample. In certain embodiments, the concentrated sample is substantially composed of steroids and the concentration of the anti-oxidant is suitably in the range of from about 0.01 percent (w/v) to about 1 percent (w/v) based on volume of the steroids.

In certain embodiments, the concentrated sample may include at least one ketolic steroid or metabolite thereof, the steroid derivatives comprising one or more derivatives of one or more steroids, one or more derivatives of one or more steroid metabolites, or any combination thereof. The hydroxyl protecting agent suitably reacts only with hydroxyl groups on the steroids. The ketolic steroids present in the sample do not require first protecting the carbonyl group with a protecting agent.

After the steroids are derivatize with the hydroxyl protecting group, the steroid derivatives are at least partially purified using a suitable procedure that separates them according to differences in their molecular characterization (e.g., molecular mass, polarity, hydrophobicity, and the like). As used herein, the phrase “at least partially” means partially, essentially completely, or completely. Accordingly, a partially purified steroids may contain some non-steroid components, for example, water, solvents, salts, molecular fragments, contaminants, and the like. Essentially completely purified steroids contain essentially no non-steroid components; non-steroid components may be present to the extent that there is no detectable difference in properties between essentially completely purified steroids and completely purified steroids. A suitable purification procedure includes a chromatographic procedure, for example liquid chromatography (LC). HPLC is preferably used.

Purification and separation of the steroid derivatives is suitably carried out using a liquid chromatography method, such as HPLC. In certain embodiments, the steroids can be reacted with a hydroxyl protecting reagent prior to, simultaneously with, or subsequent to separation by liquid or gas chromatography. Reverse-phase HPLC can also be used in certain embodiments, which is preferably performed using a gradient solution comprising a suitable alcohol and an aqueous acidic solution. A suitable alcohol is methanol and a suitable aqueous acidic solution is aqueous formic acid. A non-polar stationary phase is also suitably used, examples of which includes a C18 stationary phase. Suitable gradients include gradient elutions from about 70:30 to about 85:15, based on volume, of methanol:aqueous formic acid solution.

In certain embodiments of the present invention, a method is provided that further includes the step of adding a standard of one or more steroids to the sample, to the concentrated sample, into the purified sample prior to MS detection, or any combination thereof. The standards may be non-deuterated, but are preferably deuterated for conducting quantitative analysis on the mass spectra. Suitable deuterated standards of steroids and metabolites thereof are commercially available, such as from C/D/N Isotopes, Inc. (Pointe-Claire, Quebec, Canada), Medical Isotopes, Inc. (Pelham, N.H.). Non-deuterated standards are available from Alltech Applied Science Labs (State College, Pa., www.alltechWEB.com). Various embodiments include one, two, three, four, five, six, seven, eight, nine, ten, 11, 12, 13, 14, 15, or even 16 or more standards. Combinations or deuterated and non-deuterated standards are envisioned. Greater numbers of deuterated standards (e.g., five or more) used in certain embodiments of the methods of the present invention affords the ability to quantify a large number (e.g., eight, nine, 10, 11, 12, 13, 14, 15 or even 16 or more) of steroids.

After the steroid derivatives are suitably purified, they are analyzed using mass spectrometry (MS), along with any deuterated or non-deuterated standards. Atmospheric pressure ionization mass spectrometry, such as positive ion mode electrospray mass spectrometry, is preferably used. It is further preferred to use HPLC-ESI-MSⁿ to ascertain the amount of steroids in the samples. Procedures for analyzing steroids using MS are generally provided in U.S. Patent Appl. No. 2005/0181514, “Methods for Separation and Detection of Ketosteroids and Other Carbonyl-Containing Compounds”, by Xu, et al., the portion of which pertaining to HPLC-ESI-MS is incorporated by reference herein.

The present invention also encompasses kits for use in a method for detecting one or more steroids, such estrogens and metabolites thereof, in a biological sample by mass spectrometry. The kits comprise in packaged combination: an antioxidant, reducing agent, or any combination thereof; a deuterated standard of one or more steroids; a hydroxyl protecting reagent; and a derivatization buffer characterized as having a pH in the range of from about 7 to about 11.5. Any of the hydroxyl protecting reagents described hereinabove can be suitably included in the kits. For example, the hydroxyl protecting reagent may include a compound that forms a silyl derivative, an acyl derivative, a benzoyl derivative, an alkyl derivative, a dansyl derivative, a nitrobenzofuran derivative, or any combination thereof. In particular, the hydroxyl protecting reagent may include nitrobenzopentafluorobenzoyl hydroxylamine, hydroxylamine, dabsyl chloride, 1-fluoro-2,4-dinitrobenzene, 4-fluoro-3-nitrobenzofurazan, and preferably dansyl chloride, as well as any combination thereof.

Suitable deuterated standards in the kits include one or more deuterated estrogens, preferably one or more deuterated catechol estrogens. For example, the kits can include one or more of the following deuterated estrogen standards: d₄-E₂ as the internal standard for E₂ and E₁; d₃-E₃ for E₃, 16-KetoE₂, and 16α-OHE₁; d₃-16-epiE₃ for 16-epiE₃ and 17-epiE₃; d₅-2-MeOE₂ for 2-MeOE₂, 4-MeOE₂, 2-MeOE₁, 4-MeOE₁, and 3-MeOE₁; and d₅-2-OHE₂ for 2-OHE₂, 2-OHE₁, and 4-OHE₁. In preferred embodiments, all five of these deuterated estrogen standards are included. The deuterated standards may also be derivatized with an ionizable group for detection with mass spectrometry.

Suitable anti-oxidant in the kits include an ascorbic acid, a tocopherol, a carotenoid, beta carotene, butylated hydroxyanisole, butylated hydroxytoluene, propyl gallate, trihydroxybutyrophenone, uric acid, or any combination thereof. Preferably the antioxidant includes L-ascorbic acid. A suitable derivatization buffer includes a sodium bicarbonate buffer having a pH in the range of from about 8.5 to about 11.5.

Some embodiments of the kits of the present invention can also include a hydrolysis buffer, as described hereinabove, for deconjugating conjugated steroids. In certain embodiments, the kits of the present invention further comprising instructions for reacting the components of the kit with steroids in a biological specimen. The instructions generally indicate the steps for preparing a biological specimen, such as urine, for derivatization with the hydroxyl protecting group. The instructions may also indicate the treatment of the biological sample with any one or more of the anti-oxidant, reducing agent, hydrolyzing agent, buffer, or any combination thereof. The instructions may further indicate how to incorporate deuterated standards and perform a method to detect steroids, such as estrogens, in the biological sample.

### EXAMPLES AND ADDITIONAL ILLUSTRATIVE EMBODIMENTS

Urine Specimen Analysis. An embodiment of the present invention is exemplified using a sensitive, specific, accurate, and precise high-performance liquid chromatography-electrospray ionization-tandem mass spectrometry method (HPLC-ESI-MS²) for measuring the absolute quantities of fifteen endogenous estrogens and their metabolites in human urine has been developed and validated. This example uses only single hydrolysis, extraction, and derivatization steps and only 0.5 mL of urine, yet is capable of accurately and precisely measuring the absolute quantities of fifteen endogenous estrogens and their metabolites, including catechol, methoxy, and 16α-hydroxylated metabolites (FIG. 1), found in urines from pre- and postmenopausal women as well as men. More specifically, this method simultaneously quantified estrone and its 2-, 4-methoxy and 2-, 4-, and 16α-hydroxy derivatives, and 2-hydroxyestrone-3-methyl ether; estradiol and its 2-, 4-methoxy and 2-, 16α-hydroxy derivatives, 16-epiestriol, 17-epiestriol, and 16-ketoestradiol in pre- and postmenopausal women as well as men. Standard curves were linear over a 10³-fold concentration range with linear regression correlation coefficients typically greater than 0.996. The lower limit of quantitation for each estrogen was 0.02 ng per 0.5-mL urine sample (2 pg on column), with an accuracy of 96-107% and an overall precision, including the hydrolysis, extraction, and derivatization steps, of 1-5% relative standard deviation (RSD) for samples prepared concurrently and 1-12% RSD for samples prepared in separate batches. Since individual patterns of estrogen metabolism may influence the risk of breast cancer, the method exemplified here provides an accurate, precise, and specific measurement of endogenous estrogen metabolites in biological matrices that can facilitate the prevention, screening, and treatment of diseases (e.g., cancer), specifically breast cancer.

Reagents and Materials. Fifteen estrogen metabolites (EM) including estrone (E₁), estradiol (E₂), estriol (E₃), 16-epiestriol (16-epiE₃), 17-epiestriol (17-epiE₃), 16-ketoestradiol (16-ketoE₂), 16α-hydroxyestrone (16α-OHE₁), 2-methoxyestrone (2-MeOE₁), 4-methoxyestrone (4-MeOE₁), 2-hydroxyestrone-3-methyl ether (3-MeOE₁), 2-methoxyestradiol (2-MeOE₂), 4-methoxyestradiol (4-MeOE₂), 2-hydroxyestrone (2-OHE₁), 4-hydroxyestrone (4-OHE₁), and 2-hydroxyestradiol (2-OHE₂) were obtained from Steraloids, Inc. (Newport, R.I.). Deuterium-labeled estrogen metabolites (d-EM), including estradiol-2,4,16,16-d₄ (d₄-E₂), estriol-2,4,17-d₃ (d₃-E₃), 2-hydroxyestradiol-1,4,16,16,17-d₅ (d₅-2-OHE₂), and 2-methoxyestradiol-1,4,16,16,17-d₅ (d₅-2-MeOE₂), were purchased from C/D/N Isotopes, Inc. (Pointe-Claire, Quebec, Canada). 16-Epiestriol-2,4,16-d₃ (d₃-16-epiE₃) was obtained from Medical Isotopes, Inc. (Pelham, N.H.). All EM and d-EM analytical standards have reported chemical and isotopic purity ≧98%, and were used without further purification. Dichloromethane (HPLC grade), methanol (HPLC grade) and formic acid (reagent grade) were obtained from EM Science (Gibbstown, N.J.). Glacial acetic acid (HPLC grade), sodium bicarbonate (reagent grade), and L-ascorbic acid (reagent grade) were purchased from J. T. Baker (Phillipsburg, N.J.) and sodium hydroxide (reagent grade) and sodium acetate (reagent grade) were purchased from Fisher Scientific (Fair Lawn, N.J.). β-Glucuronidase/sulfatase from Helix pomatia (Type H-2) was obtained from Sigma Chemical Co. (St. Louis, Mo.), and dansyl chloride (reagent grade), p-toluenesulfonhydrazide (reagent grade), and acetone (HPLC grade) were purchased from Aldrich Chemical Co. (Milwaukee, Wis.).

Urine Sample Collection. First-morning urine samples were collected in 1 L bottles containing 1 g ascorbic acid (to prevent oxidation) from ten premenopausal women (aged from 28-47 years, average 33.7 years), ten postmenopausal women (aged from 53-69 years, average 58.7 years), and five men (aged from 30-39 years, average 32.8 years). All subjects were healthy, non-pregnant, and none of them was taking exogenous hormones. The urine samples obtained from the premenopausal women were collected during both follicular and luteal phase of the menstrual cycle. Immediately after collection, the volumes of the urine samples were recorded. Aliquots of urines were stored at −80° C. prior to analysis.

Preparation of Stock and Working Standard Solutions. Stock solutions of EM and d-EM were each prepared at 80 μg/mL by dissolving 2 mg of the estrogen powders in methanol to a final volume of 25 mL in a volumetric flask and stored at −20° C. During each day of analysis, working standards of EM and d-EM were prepared by dilutions of the stock solutions using methanol with 0.1% L-ascorbic acid. The EM and d-EM working standard solutions were prepared at 80 ng/mL.

Preparation of Calibration Standards and Quality Control Samples. Charcoal stripped human urine (Golden West Biologicals, Temecula, Calif.) that contains 0.1% (w/v) L-ascorbic acid and has no detectable levels of EM was employed for preparation of calibration standards and quality control samples. Calibration standards were prepared in charcoal stripped human urine by adding 20 μL of the d-EM working internal standard solution (1.6 ng d-EM) and various volumes of EM working standard solution, which typically contained from 0.02 to 19.2 ng EM. Quality control samples were also prepared in charcoal stripped human urine at three levels (0.12, 0.96, and 6.4 ng EM per mL).

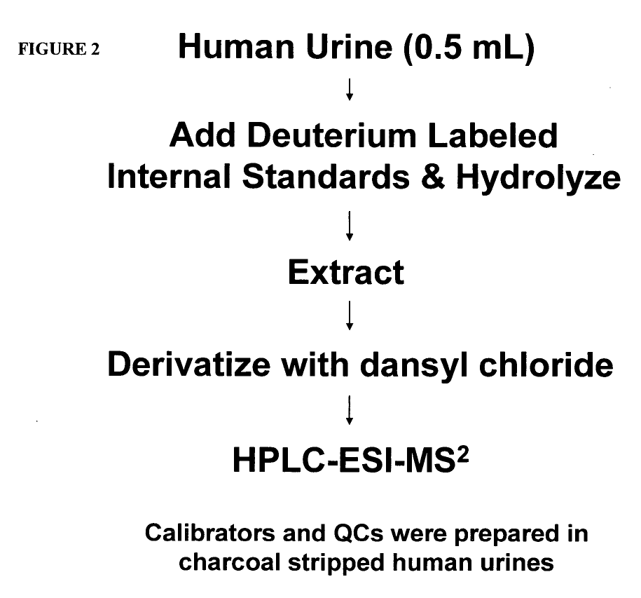

Hydrolysis and Extraction Procedure. The general procedure for the measurement of EM used in this example is depicted schematically in FIG. 2. Since endogenous estrogens and their metabolites in urine are mostly present as glucuronide conjugates and small amounts of sulfate conjugates, a hydrolysis step was included to deconjugate them. To a 0.5 mL aliquot of urine, 20 μL of the d-EM working internal standard solution (1.6 ng d-EM) was added, followed by 0.5 mL of freshly prepared enzymatic hydrolysis buffer containing 2 mg of L-ascorbic acid, 5 μL of β-glucuronidase/sulfatase from Helix pomatia (Type H-2) and 0.5 mL of 0.15 M sodium acetate buffer (pH 4.1). The sample was incubated overnight at 37° C. After hydrolysis, the sample was subjected to slow inverse extraction at 8 rpm (RKVSD™, ATR, Inc., Laurel, Md.) with 7 mL dichloromethane for 30 min. After extraction, the aqueous layer was discarded and the organic solvent portion was transferred into a clean 16×125 mm glass tube and evaporated to dryness at 55° C. under nitrogen gas (Reacti-Vap III™, Pierce, Rockford, Ill.).

Derivatization Procedure. To the dried sample 100 μL of 0.1 M sodium bicarbonate buffer (pH at 9.0) and 100 μL of dansyl chloride solution (1 mg/mL in acetone) were added. After vortexing, the sample was heated at 60° C. (Reacti-Therm III™ Heating Module, Pierce, Rockford, Ill.) for 5 min to form the EM and d-EM dansyl derivatives (EM-Dansyl and d-EM-Dansyl, respectively). Calibration standards and quality control samples were hydrolyzed, extracted, and derivatized following the same procedure as that of unknown urine samples. After derivatization, all samples were analyzed by HPLC-ESI-MS².

High Performance Liquid Chromatography-Electrospray Ionization Tandem Mass Spectrometry Analysis (HPLC-ESI-MS²). HPLC-ESI-MS² analysis was performed using a Finnigan TSQ™ Quantum-AM triple quadrupole mass spectrometer coupled with a Surveyor HPLC system (ThermoFinnigan, San Jose, Calif.). Both the HPLC and mass spectrometer were controlled by Xcalibur™ software (ThermoFinnigan). Liquid chromatography was carried out on a 150 mm long×2.0 mm i.d. column packed with 4 μm Synergi Hydro-RP particles (Phenomenex, Torrance, Calif.) maintained at 40° C. A total of 20 μL of each sample was injected onto the column. The mobile phase, operating at a flow rate of 200 μL/min, used methanol as solvent A and 0.1% (v/v) formic acid in water as solvent B. For the analysis of EM-Dansyl and d-EM-Dansyl, a linear gradient changing the A/B solvent ratio from 72:28 to 85:18 in 75 min was employed. After washing with 100% A for 12 min, the column was re-equilibrated with a mobile phase composition A/B of 72:28 for 13 min prior to the next injection. The general MS conditions were as follows: source: ESI; ion polarity: positive; spray voltage: 4600 V; sheath and auxiliary gas: nitrogen; sheath gas pressure: 49 arbitrary units; auxiliary gas pressure: 23 arbitrary units; ion transfer capillary temperature, 350° C.; scan type: selected reaction monitoring (SRM); collision gas: argon; collision gas pressure: 1.5 mTorr. The SRM conditions for the protonated molecules [MH⁺] of EM-Dansyl and d-EM-Dansyl were as following: E₁ m/z 504→171 collision energy: 42 eV; E₂ m/z 506→171 collision energy: 43 eV; E₃, 16-epi E₃, and 17-epi E₃ m/z 522→171 collision energy: 43 eV; 16-KetoE₂, and 16α-OHE, m/z 520→171 collision energy: 43 eV; 2-MeOE₁, 4-MeOE₁, and 3-MeOE₁, m/z 534→171 collision energy: 42 eV; 2-MeOE₂ and 4-MeOE₂ m/z 536→171 collision energy: 43 eV; 2-OHE₁ and 4-OHE₁ m/z 753→170 collision energy: 44 eV; 2-OHE₂ m/z 755→170 collision energy: 43 eV; d₄-E₂ m/z 510→171 collision energy: 43 eV; d₃-E₃ and d₃-16-epiE₃ m/z 525→171 collision energy: 43 eV; d₅-2-MeOE₂ m/z 541→171 collision energy: 43 eV; d₅-2-OHE₂ m/z 760→170 collision energy: 43 eV. The following MS parameters were used for all experiments, scan width: 0.7 u; scan time: 0.50 s; Q1 peak width: 0.70 u FWHM; Q3 peak width: 0.70 u FWHM.

Quantitation of Estrogen Metabolites (EM). Quantitation of EM in urine was carried out using Xcalibur™ Quan Browser (ThermoFinnigan). Calibration curves for the fifteen EM were constructed by plotting EM-Dansyl/d-EM-Dansyl peak area ratios obtained from calibration standards versus amounts of EM and fitting these data using linear regression with 1/X weighting. The amount of EM in urine samples was then interpolated using this linear function. Only the deuterium labeled standards without exchange loss were employed in this study, d₄-E₂ was used as the internal standard for E₂ and E₁; d₃-E₃ for E₃, 16-KetoE₂, and 16α-OHE₁; d₃-16-epiE₃ for 16-epiE₃ and 17-epiE₃; d₅-2-MeOE₂ for 2-MeOE₂, 4-MeOE₂, 2-MeOE₁, 4-MeOE₁, and 3-MeOE₁; d₅-2-OHE₂ for 2-OHE₂, 2-OHE₁, and 4-OHE₁.

Absolute Recovery of Estrogen Metabolites after Hydrolysis and Extraction Procedure. To one set of six 0.5-mL aliquots of the charcoal stripped human urine, 20 μL of the EM working standard solution (1.6 ng EM) was added, followed by the hydrolysis and extraction procedure described above. A second set of six 0.5-mL aliquots of the charcoal stripped human urine was treated identically, except that the EM was added after the hydrolysis and extraction procedure. Both sets of samples were derivatized and analyzed in consecutive LC-MS analyses. The absolute recovery of EM after the hydrolysis and extraction procedure was calculated by dividing the mean of EM-Dansyl peak area from the second set into that from the first set.

Accuracy and Precision of the Urinary Estrogen Metabolite Analysis. To assess the accuracy and precision of our method, four replicated 0.5 mL aliquots of 0.12, 0.96, and 6.4 ng/mL control urine samples were hydrolyzed, extracted, derivatized, and analyzed in four different batches. The accuracy was measured as the percent matching of calculated amount to known amount of EM in control urine samples. The intra- and inter-batch precisions were measured by the percent relative standard deviations.

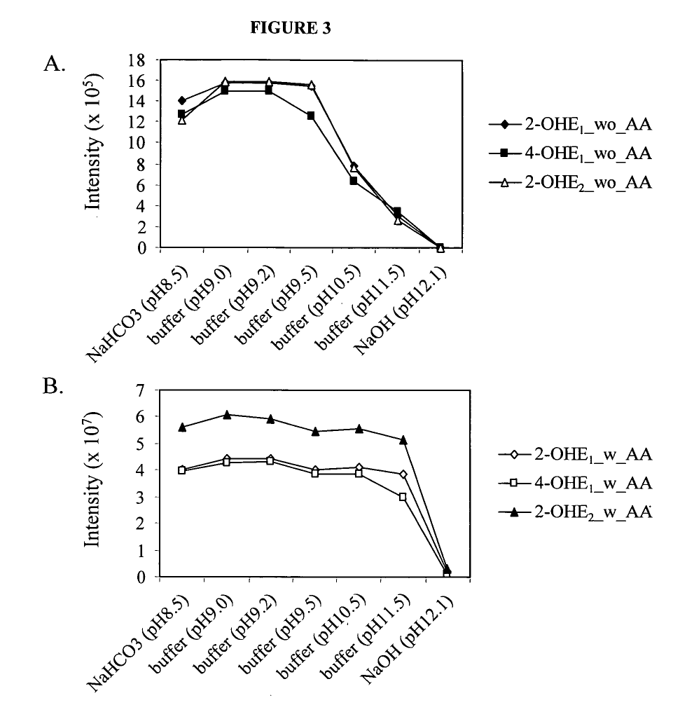

Results: Optimal Conditions for Estrogen Metabolite Derivatization. The levels of endogenous estrogens and their metabolites are quite low (typically in the pg/mL range) depending on the sex, age, and menopausal status of the patient. Therefore, the effects of reaction heating time and temperature, dansyl chloride concentration, pH, and presence of L-ascorbic acid upon the yield of dansylation starting from the same amount of EM were carefully examined to yield to best conditions for derivatizing EM. When other conditions were the same, heating sample at 60° C. for 5 min gave the best yield of dansylation for all EM. Increasing dansyl chloride concentration from 1 mg per mL to 3 mg per mL did not improve the yield of dansylation under the same conditions. No significant change in the extent of dansylation for non-catechol estrogens at pH 8.5-11.5 in the presence or absence of 0.1% (w/v) L-ascorbic acid was observed. The presence of 0.1% (w/v) L-ascorbic acid did, however, result in a significant (up to 100 fold) increase in the dansylation efficiency of catechol estrogens (FIG. 3). Therefore, 0.1% (w/v) L-ascorbic acid was used in all samples including all calibration standards, quality controls, and unknown human urines.

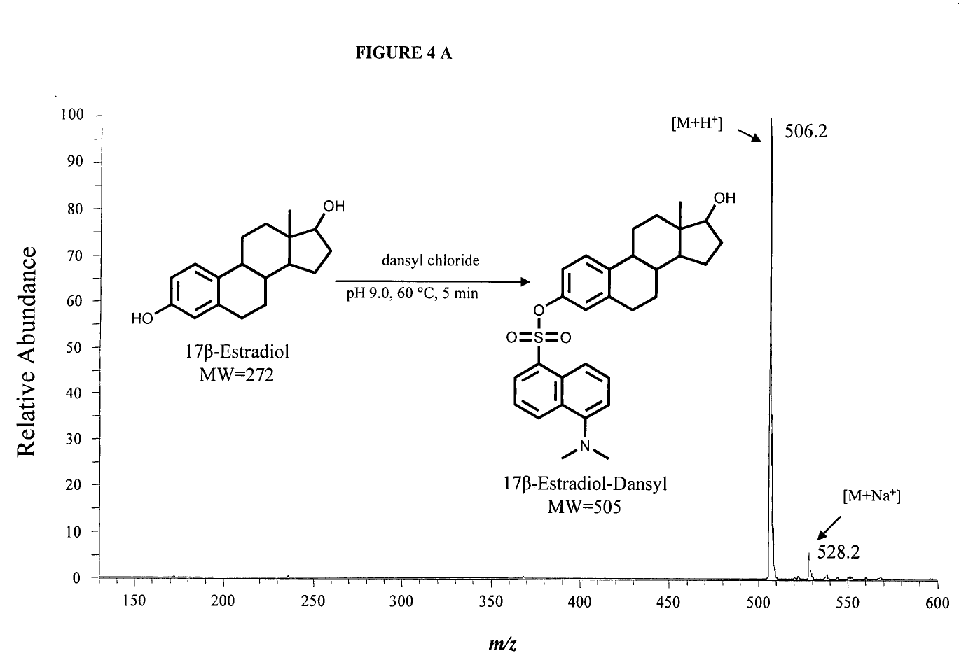

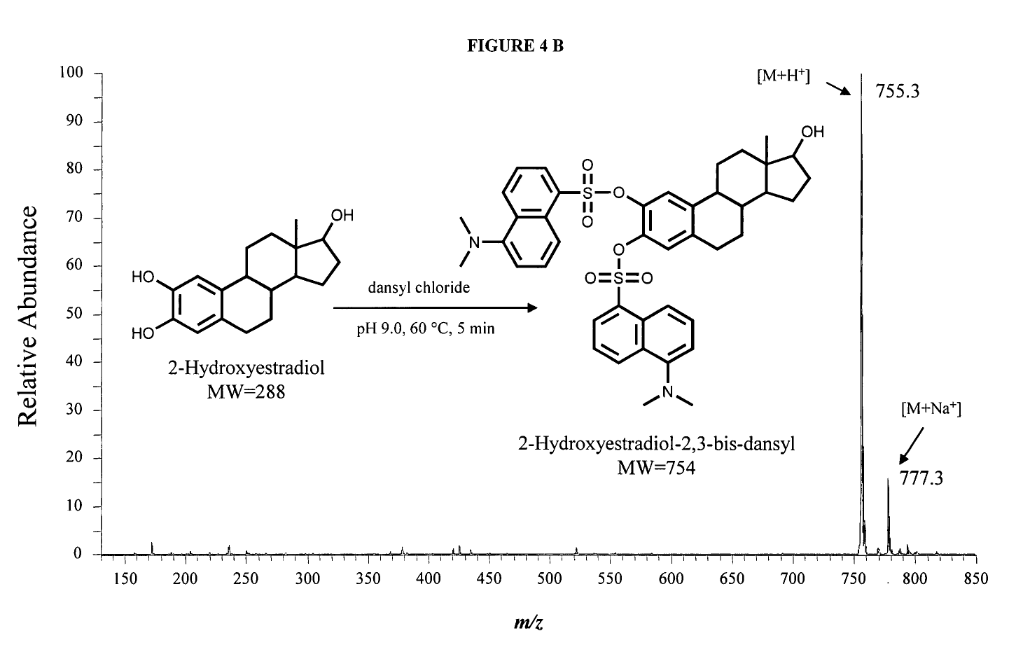

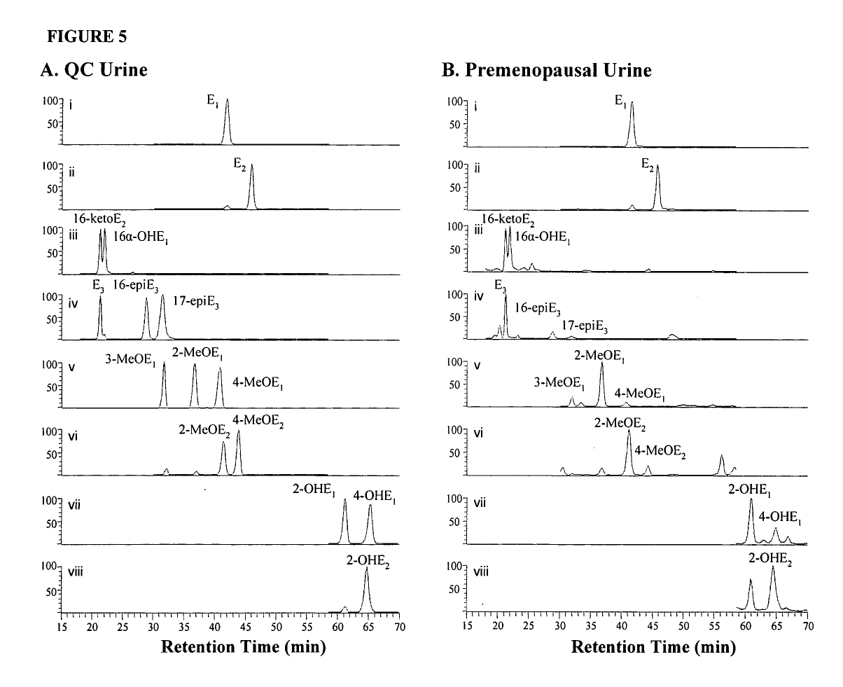

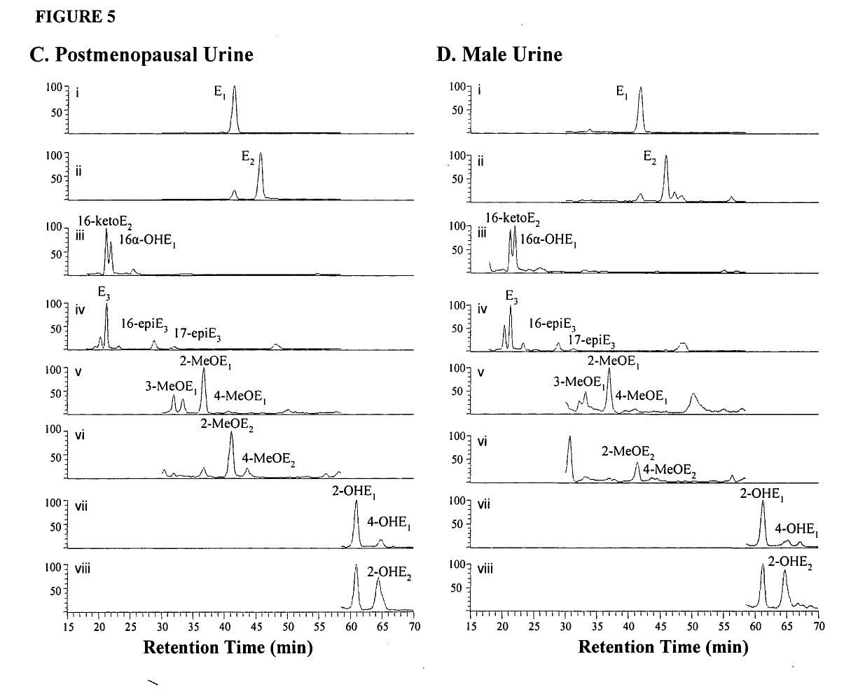

Mass Spectral and Chromatographic Profiles of Estrogens in Quality Control and Pooled Human Urines. The MS full scans of EM-Dansyl and d-EM-Dansyl are characterized by an intense protonated molecule [MH⁺], and a much less abundant sodiated molecule [MNa⁺] (FIGS. 4A and B). The major ion in the [MH⁺] product ion full scan is observed at m/z 170 for catechol estrogens and m/z 171 for the remaining estrogens and estrogen metabolites. The HPLC-ESI-MS² selected reaction monitoring (SRM) chromatographic profiles of a 0.12-ng EM/mL urine quality control sample, a pooled premenopausal urine sample, a pooled postmenopausal urine sample, and a pooled male urine sample are shown in FIGS. 5A-D, respectively. Using a methanol-water linear gradient, all fifteen EM were separated by reversed phase C₁₈ chromatography within a 70-min time range, and gave symmetrical peak shapes, which facilitates accurate quantitative measurements. Even though only a single hydrolysis, extraction, and derivatization steps and 0.5 mL human urine sample was used, this method was adequate to quantitatively measure fifteen endogenous estrogens and estrogens metabolites in all of the urine samples, even those obtained from men and postmenopausal women.

Calibration Curve and Limit of Quantitation. An important consideration in the development of any assay is the linearity range and sensitivity of the assay. The sheer number of EM being measured in this study span a wide range of concentrations between the various types of samples being analyzed. The calibration curves (FIGS. 5A-5H) for the detection of each EM were linear over an approximately 10³-fold range of concentration (0.02-19.2 ng/sample or 0.04-38.4 ng/mL) with correlation coefficients for the linear regression curves typically greater than 0.996. The signal-to-noise (S/N) ratios obtained from the 0.02-ng calibration standard, representing 2 pg EM on column, were typically greater than 200, and intra- and inter-batch precision was consistently within 5 and 15% RSD, respectively. These levels of detection and precision demonstrate the utility of using this analytical approach for quantitatively measuring endogenous estrogens and their metabolites in urines from a wide range of patients including postmenopausal women and men. These methods can easily be extended by one of ordinary skill in the art to quantitatively measuring endogenous estrogens and their metabolites in other biological samples, such as tissue, blood, plasma and serum. These methods can also be easily be extended by one of ordinary skill in the art to quantitatively measuring other steroids and metabolites thereof besides estrogens, such as androgens and progestines.

Absolute Recovery of Estrogen Metabolites after the Hydrolysis and Extraction Procedure. Since the concentrations of endogenous estrogen and metabolites thereof can range into the pg/mL levels in samples obtained from postmenopausal women and men, the sample processing procedure desirably retains a high percentage of the starting material. The absolute recovery of EM after the hydrolysis and extraction procedure was determined by comparing chromatographic peak area of EM-Dansyl in charcoal stripped human urine that had been spiked with EM before and after the hydrolysis and extraction procedure. Using this method, the mean absolute recoveries ranged from 86.3 to 93.6%. This high level of recovery not only demonstrates the high sensitivity of this method, but it also demonstrates the overall precision and accuracy of this method.

Accuracy and Precision of the Urinary EM Analysis. To measure the accuracy and intra-batch precision of this method, four replicated 0.5 mL aliquots of 0.12, 0.96, and 6.4 ng/mL control urine samples were hydrolyzed, extracted, derivatized, and analyzed by HPLC-ESI-MS². As shown in Table 1, the accuracy of the measurements of the 0.12, 0.96, and 6.4 ng/mL samples range from 98-106%, 96-103%, and 97-107%, respectively. The intra-batch precision, as estimated by the RSD from 4 replicate urine analyses at each concentration level, was 2-5%, 1-5%, and 1-3% for the 0.12, 0.96, and 6.4 ng/mL control urine samples, respectively. Inter-batch precision data for the analysis of urinary EM, including hydrolysis, extraction, derivatization, and HPLC-ESI-MS² steps, is presented in Table 2. The inter-batch precision of EM measurement estimated by the RSD from four independent batch analyses ranged from 4-12%, 1-7%, and 1-5% for 0.12, 0.96, and 6.4 ng/mL control urine samples, respectively.

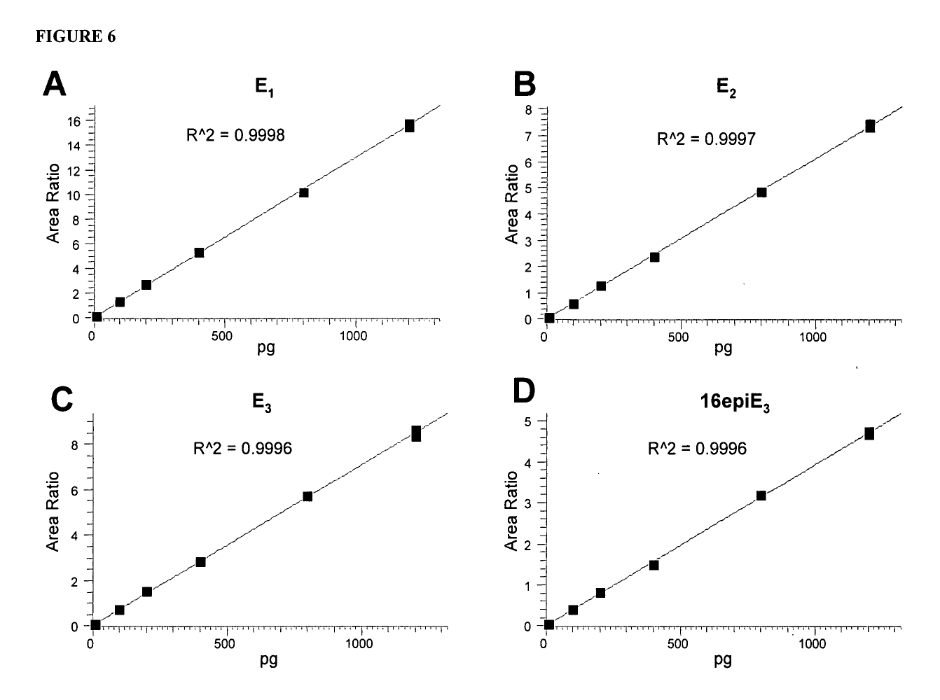

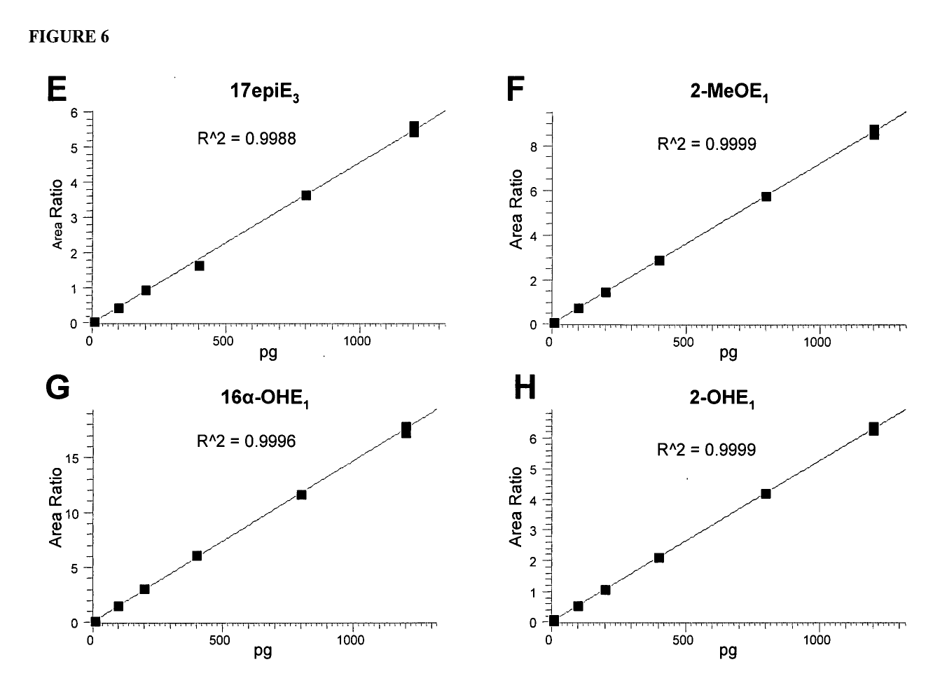

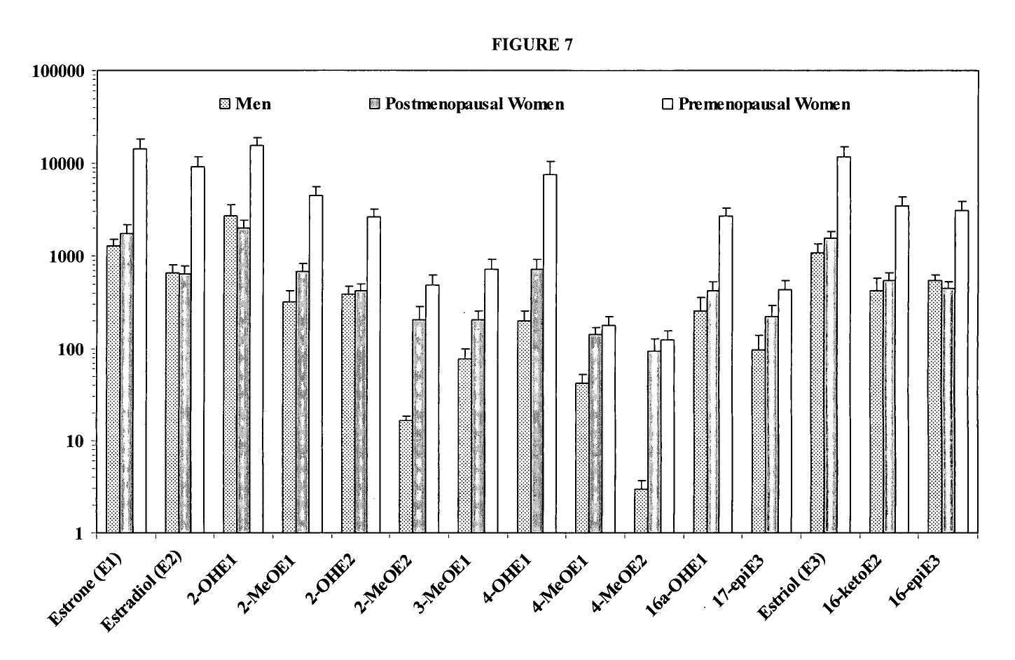

Application to Pre- and Postmenopausal and Male Urine Samples. To test its utility for quantitatively measuring estrogen metabolites in actual clinical samples, urine samples from ten postmenopausal women, ten premenopausal women, and five men were analyzed using the described method. Duplicate 0.5-mL aliquots from each urine sample were hydrolyzed, extracted, derivatized, and analyzed by HPLC-ESI-MS² to determine individual EM concentrations (FIGS. 6-7). According to these results, premenopausal women excreted much greater amount of estrogens and estrogen metabolites than postmenopausal women and men. In addition to the parent estrogens, E₁ and E₂, humans excreted great amount of estrogen metabolites as catechol estrogens such as 2-OHE₁ and 4-OHE₁, from 16α-hydroxylation such as E₃, 16α-OHE₁, and 16-ketoE₂, and as methoxy estrogens such as 2-MeOE₁. Significant inter-individual variation has been observed even within the same group such as among postmenopausal women, premenopausal women, or men. Although in most cases the amount of 2-OHE₁ excretion is greater than 4-OHE₁, the opposite was observed in one premenopausal and one postmenopausal women. Epidemiology studies can be conducted to examine the impact of these variations upon diseases, such as breast cancer risk. This study is the first to provide a detailed measurement of the levels of EM in men. Accordingly, the methods disclosed herein can be used in studying and diagnosing male hormone related cancers as well.

With mounting evidence that endogenous estrogens and their metabolites play a role in the development of breast cancer and that women with high circulating and urinary estrogen levels are at an increased risk, the methods disclosed herein for providing a sensitive, specific, accurate, and precise assay capable of measuring individual endogenous estrogens and estrogen metabolites in various biological matrices is an important development. The present methods enable an epidemiological study in which hundreds of clinical samples are analyzed in understanding the connection between estrogen and estrogen metabolite levels and the presence of a disease, or the likelihood of developing a disease, such as breast cancer. The present methods therefore provide distinct advantages over previously available assays that are either too non-specific or laborious. In addition, the present methods are capable of efficiently measuring a wide range of both ketolic and non-ketolic estrogen metabolites, which is otherwise not possible using previous assays.

The exemplified embodiment of the present invention provides a sensitive, specific, accurate, precise, and high-throughput HPLC-ESI-MS² method for simultaneously measuring 15 endogenous estrogens and estrogen metabolites in urines from pre- and postmenopausal women and from men. Compared to previous stable isotope dilution/GC-MS methods, this method greatly simplifies the sample preparation procedure resulting in a high-throughput analytical method that is suitable for epidemiology studies. Standard curves were linear over a 10³-fold concentration range (0.02-19.2 ng EM/sample), with linear regression correlation coefficients typically greater than 0.996. The lower limit of quantitation for each EM is 0.02 ng per 0.5-mL urine sample, with an accuracy of 96-107% and an overall precision of 1-5% for samples prepared concurrently and 1-12% for samples prepared in several batches. Accordingly, the method described herein can be used in a number of applications, such as (i) in epidemiology studies to determine the link between EM levels and breast cancer risk; (ii) in determining the presence of, or the likelihood of contracting, a disease or condition in a mammal; (iii) the presence of illegal steroids in a mammal; and other applications of the like in which the determination of a steroid profile of a mammal is needed.

TABLE 1
Accuracy and intra batch precision of urinary estrogen metabolite measurement, including
hydrolysis, extraction, derivatization, and LC-MS steps^{a}.

| | 0.12 ng per mL urine | | 0.96 ng per mL urine | | 6.4 ng per mL urine | |
|---|---|---|---|---|---|---|
| | Accuracy | | Accuracy | | Accuracy | |
| Estrogen | (%) | Precision (%) | (%) | Precision (%) | (%) | Precision (%) |
| E₁ | 102.9 | 3.1 | 99.9 | 4.8 | 98.9 | 1.8 |
| E₂ | 103.4 | 3.4 | 98.9 | 3.2 | 97.6 | 1.9 |
| 16α-OHE₁ | 102.6 | 4.4 | 103.2 | 3.6 | 106.6 | 1.6 |
| 16-ketoE₂ | 99.5 | 5.1 | 103.0 | 1.8 | 106.1 | 2.9 |
| E₃ | 104.8 | 3.8 | 103.2 | 2.5 | 107.5 | 2.5 |
| 16-epiE₃ | 98.2 | 4.3 | 96.2 | 3.0 | 102.2 | 2.9 |
| 17-epiE₃ | 102.6 | 3.3 | 95.6 | 3.1 | 96.7 | 1.9 |
| 2-OHE₁ | 105.1 | 3.7 | 101.3 | 3.3 | 102.6 | 2.6 |
| 2-OHE₂ | 106.1 | 3.3 | 103.3 | 1.3 | 102.7 | 1.3 |
| 4-OHE₁ | 106.2 | 4.5 | 100.8 | 2.9 | 102.2 | 2.3 |
| 2-MeOE₁ | 102.6 | 3.0 | 96.2 | 4.9 | 97.2 | 1.8 |
| 2-MeOE₂ | 103.2 | 2.3 | 97.2 | 2.2 | 96.8 | 1.2 |
| 3-MeOE₁ | 106.4 | 4.4 | 99.7 | 3.4 | 98.4 | 2.9 |
| 4-MeOE₁ | 101.1 | 2.1 | 97.4 | 3.1 | 98.3 | 1.6 |
| 4-MeOE₂ | 105.9 | 3.3 | 98.2 | 2.8 | 99.9 | 2.5 |

^{a}The accuracy was measured as the percent matching of calculated amount to known amount of EM in control urine samples. The intra batch precisions were measured as the percent relative standard deviations.

TABLE 2
Inter-batch precision of urinary estrogen metabolite
measurement, including hydrolysis, extraction,
derivatization, and LC-MS steps^{a}.

| | Concentration | | |
|---|---|---|---|
| | 0.12 ng per mL | 0.96 ng per mL | 6.4 ng per mL |
| Estrogen | urine | urine | urine |
| E₁ | 8.0 | 2.1 | 1.6 |
| E₂ | 7.3 | 5.0 | 1.7 |
| 16α-OHE₁ | 12.1 | 6.0 | 1.1 |
| 16-ketoE₂ | 8.2 | 5.6 | 2.1 |
| E₃ | 7.6 | 3.4 | 4.0 |
| 16-epiE₃ | 3.8 | 3.2 | 2.5 |
| 17-epiE₃ | 6.2 | 3.3 | 0.7 |
| 2-OHE₁ | 5.7 | 4.5 | 4.6 |
| 2-OHE₂ | 4.4 | 1.3 | 1.3 |
| 4-OHE₁ | 7.7 | 5.2 | 2.2 |
| 2-MeOE₁ | 6.2 | 3.5 | 2.0 |
| 2-MeOE₂ | 7.0 | 3.7 | 1.9 |
| 3-MeOE₁ | 9.0 | 6.5 | 1.8 |
| 4-MeOE₁ | 5.1 | 4.7 | 2.3 |
| 4-MeOE₂ | 4.9 | 1.5 | 1.8 |

^{a}The inter batch precisions were measured as the percent relative standard deviations.

Serum Specimen Analysis. This example provides an HPLC-ESI-MS² method using 0.5 mL of serum that is capable of accurately and precisely measuring the absolute quantities of unconjugated and conjugated endogenous estrogens and estrogen metabolites. Catechol, methoxy, and 16a-hydroxylated metabolites (FIG. 1), found in sera from both pre- and postmenopausal women were analyzed. Reagents and materials as described in the above examples using human urine were used in this example with serum. The instrumentation for conducting HPLC-ESI-MS² as described above was also used in analyzing serum samples.

Serum Sample Collection. Serum samples were collected from two premenopausal women during their follicular and luteal phases of menstrual cycle, respectively, and two postmenopausal women. All subjects were healthy, non-pregnant, and none of them was using exogenous hormones. Immediately after collection, aliquots of sera were stored at −80″C prior to analysis. Protocol of our study was approved by the NCI/NIH Institutional Review Board.

Preparation of Stock and Working Standard Solutions. Stock solutions of EM and d-EM were each prepared at 80 μg/mL by dissolving 2 mg of the estrogen powders in methanol with 0.1% L-ascorbic acid to a final volume of 25 mL in a volumetric flask and stored at −20° C.; they were stable for at least 2 months, and new stock solutions were prepared after that. At the beginning of each analysis, samples of stock solutions were analyzed to verify that EM and d-EM standards gave the same results as they were freshly prepared. Working standards of EM and d-EM at 8 ng/mL were then prepared by dilutions of the stock solutions using methanol with 0.1% L-ascorbic acid.

Preparation of Calibration Standards. Charcoal stripped human serum (Golden West Biologicals, Temecula, Calif.) that contains 0.1% (w/v) L-ascorbic acid and has no detectable levels of EM was employed for preparation of calibration standards and quality control samples. Calibration standards were prepared in charcoal stripped human serum by adding 20 μL of the d-EM working internal standard solution (0.16 ng d-EM) and various volumes of EM working standard solution, which typically contained from 0.002 to 1.92 ng EM.

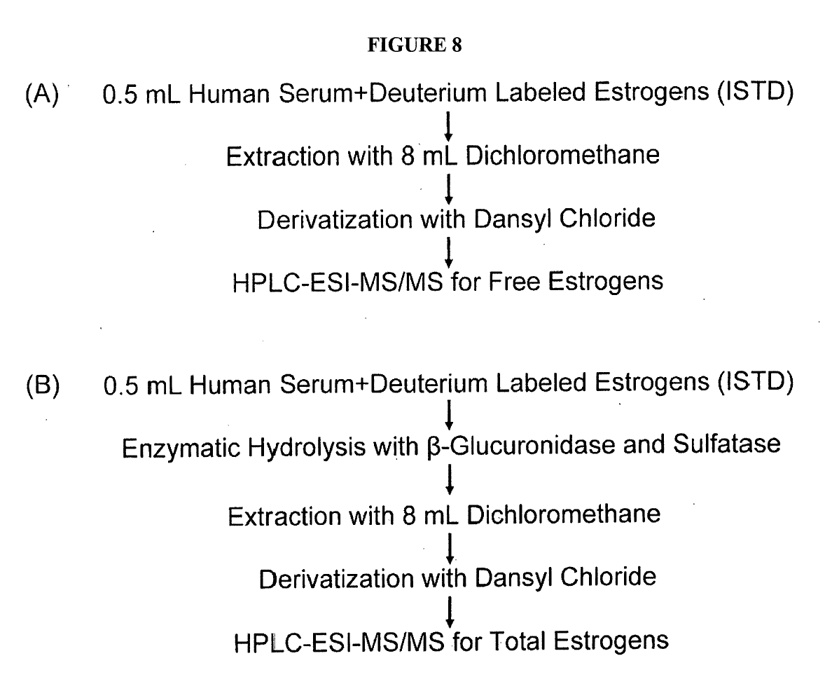

Hydrolysis and Extraction Procedure. The overall procedure for the measurement of unconjugated only and unconjugated+conjugated serum EM is shown schematically in FIG. 8B. For measuring unconjugated+conjugated serum EM: To a 0.5 mL aliquot of serum, 20 μL of the d-EM working internal standard solution (0.16 ng d-EM) was added, followed by 0.5 mL of freshly prepared enzymatic hydrolysis buffer containing 2 mg of L-ascorbic acid, 5 μL of β-glucuronidase/sulfatase from Helix pomatia (Type HP-2) and 0.5 mL of 0.15 M sodium acetate buffer (pH 4.1). The sample was incubated 20 hours at 37° C. After hydrolysis, the sample underwent slow inverse extraction at 8 rpm (RKVSD™, ATR, Inc., Laurel, Md.) with 7 mL dichloromethane for 30 min. After extraction, the aqueous layer was discarded and the organic solvent portion was transferred into a clean 16×125 mm glass tube and evaporated to dryness at 55° C. under nitrogen gas (Reacti-Vap III™, Pierce, Rockford, Ill.). For measuring unconjugated serum EM only, the same sample preparation without β-glucuronidase/sulfatase hydrolysis step was employed, as shown in FIG. 8A.

Derivatization Procedure. To the dried sample 100 μL of 0.1 M sodium bicarbonate buffer (pH at 9.0) and 100 μL of dansyl chloride solution (1 mg/mL in acetone) were added. After vortexing, the sample was heated at 60° C. (Reacti-Therm III™ Heating Module, Pierce, Rockford, Ill.) for 5 min to form the EM and d-EM dansyl derivatives (EM-Dansyl and d-EM-Dansyl, respectively). Calibration standards and quality control samples were hydrolyzed, extracted, and derivatized following the same procedure as that of unknown serum samples. After derivatization, all samples were analyzed by HPLC-ESI-MS₂.

Quantitation of Estrogen Metabolites (EM). Quantitation of serum EM was carried out using Xcalibur™ Quan Browser (ThermoFinnigan). Calibration curves for the fifteen EM were constructed by plotting EM-Dansyl/d-EM-Dansyl peak area ratios obtained from calibration standards versus amounts of EM and fitting these data using linear regression with 1/X weighting. The amount of EM in serum samples was then interpolated using this linear function. Deuteriums at α-position to the carbonyl group of the labeled ketolic estrogens were especially susceptible to exchange loss during sample preparation and analysis. To ensure the quality of quantitative analyses, only deuterium labeled estrogen standards without exchange loss were employed in this study. Based on their similarity of structures and retention times, d₄-E₂ was used as the internal standard for E₂ and E₁; d₃-E₃ for E₃, 16-KetoE2, and 16α-OHE₁; d₃-16-epiE₃ for 16-epiE₃ and 17-epiE₃; d₅-2-MeOE₂ for 2-MeOE₂, 4-MeOE₂, 2-MeOE₁, 4-MeOE₁, and 3-MeOE₁; d₅-2-OHE₂ for 2-OHE₂, 2-OHE₁, and 4-OHE₁.

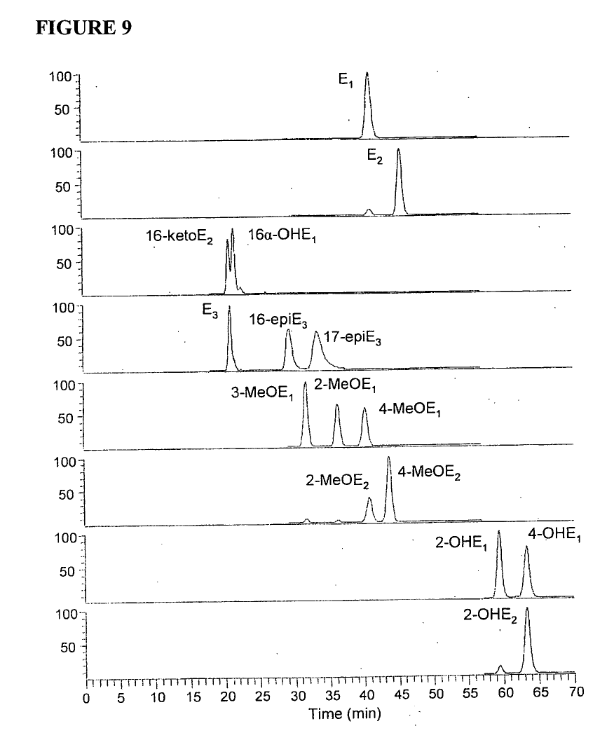

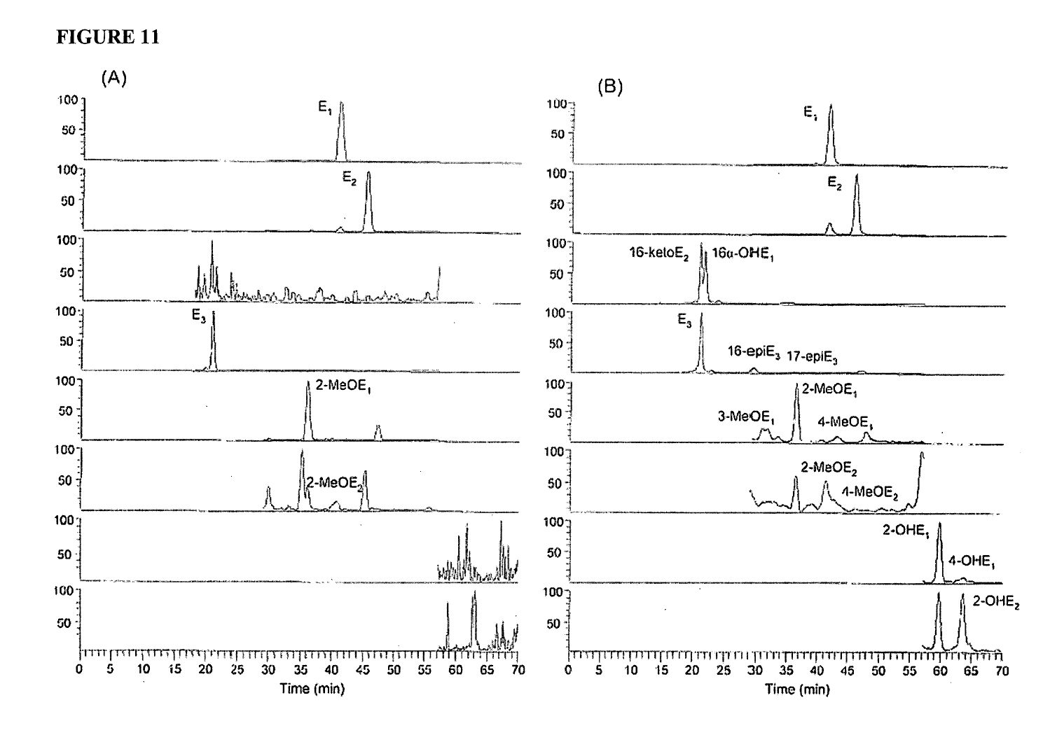

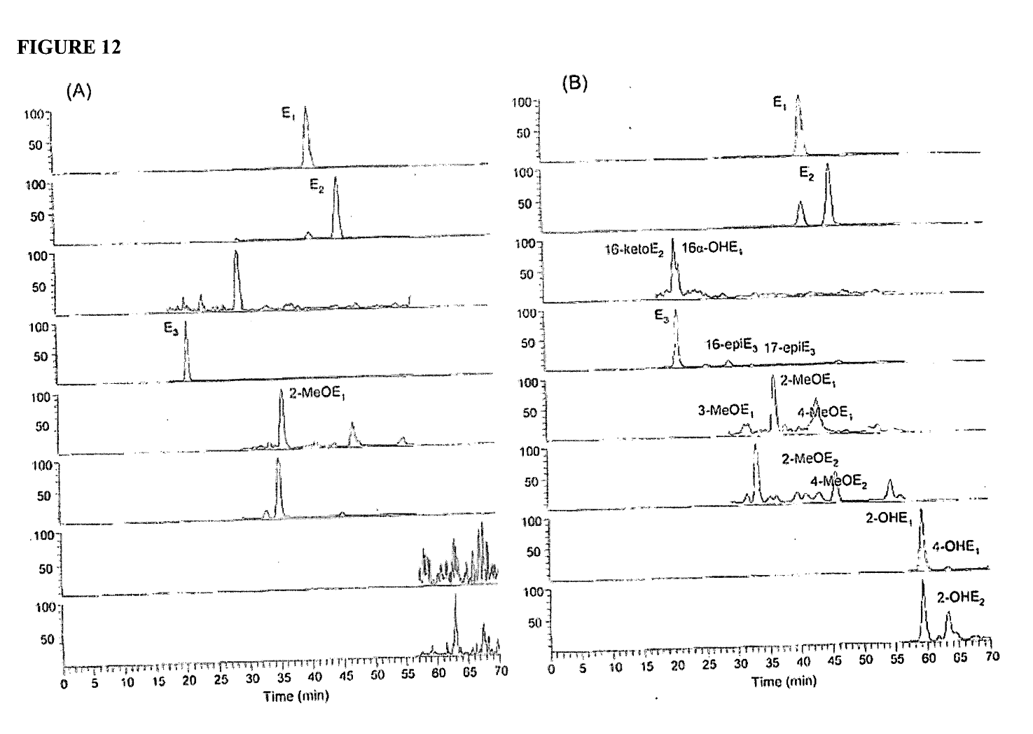

Serum Sample Results. The HPLC-ESI-MS² selected reaction monitoring (SRM) chromatographic profiles from a 0.08-ng EM/mL serum calibration sample, a premenopausal woman luteal phase serum, a premenopausal woman follicular phase serum, and a postmenopausal woman serum are shown in FIGS. 9-12, respectively. Using a methanol-water linear gradient, all fifteen EM were separated by reversed phase C₁₈ chromatography within a 70-min time range, and gave symmetrical peak shapes. These methods were capable of simultaneously quantifying five unconjugated estrogens: 17β-estradiol, estrone, estriol, 2-methoxyestrone, and 2-methoxy-17β-estradiol; as well as fifteen unconjugated+conjugated estrogens: estrone and its 2-, 4-methoxy and 2-, 4-, and 16α-hydroxy derivatives, and 2-hydroxyestrone-3-methyl ether; estradiol and its 2-, 4-methoxy and 2-, 16α-hydroxy derivatives, 16-epiestriol, 17-epiestriol, and 16-ketoestradiol in premenopausal follicular and luteal as well as postmenopausal serum samples. Calibration curves are linear over a 10³-fold concentration range with linear regression correlation coefficients typically greater than 0.996. The lower limit of quantitation for each estrogen is 0.1 pg on column with good accuracy and precision.

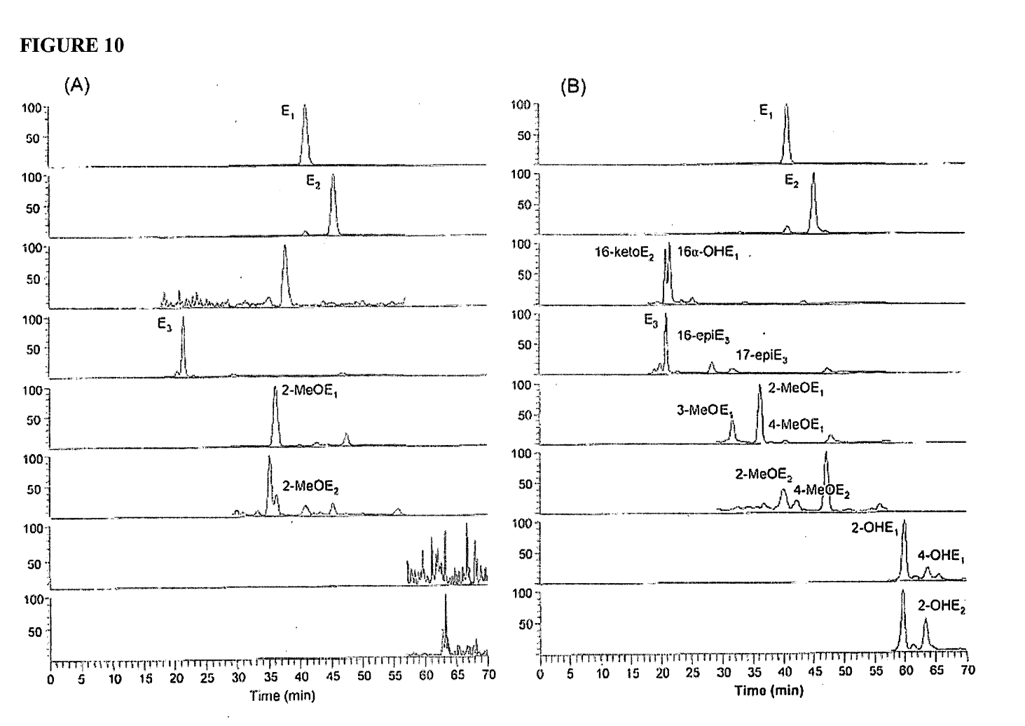

Unconjugated 17β-estradiol (E₂), estrone (E₁), estriol (E₃), 2-methoxyE₁, and 2-methoxyE₂ were found in luteal, follicular, and postmenopausal sera (FIGS. 10-12, Table 3). No unconjugated catechol estrogens or 16α-hydroxyE₁, and the like were detected. With β-glucuronidase/sulfatase hydrolysis (unconjugated+conjugated EM), concentrations of E₁, E₂, E₃, 2-methoxyE₁, and 2-methoxyE₂ were increased, especially E₁ by at least 10-fold (Table 3). In addition, catechol estrogens (2-hydroxyE₁, 4-hydroxyE₁, and 2-hydroxyE₂) and their methylated derivatives, 16α-hydroxyE₁, 16-keto-E₂, 16-epiE₃ and 17-epiE₃ were identified and quantitatively measured in luteal, follicular, and postmenopausal sera (FIGS. 10-12 and Table 3). These results indicate that reactive and harmful unconjugated endogenous estrogen metabolites such as catechol estrogens and 16α-hydroxyE₁ were in extremely low level and below the detection limit in pre- and postmenopausal woman sera. In contrast, the majority of endogenous circulating estrogens and estrogen metabolites are kept in their methoxy, sulfate or/and glucuronide forms which were known to be stable and benign. Therefore, it appears that human tissues may regulate their local unconjugated and conjugated estrogen metabolite profiles and concentrations via the control of expressions and activities of related conjugation and de-conjugation enzymes. The information obtained by these methods helps to facilitate breast cancer prevention, screening, and treatment.

TABLE 3
Serum Estrogen Concentrations (pg/mL)

| Sample Name | 16KE2 | E3 | 16aE1 | 16epiE3 | 17epiE3 | 3ME1 | 2ME1 | 4ME1 | 2ME2 |
|---|---|---|---|---|---|---|---|---|---|
| LP_1_total | 25.4 | 45.2 | 13.4 | 6.7 | 2.6 | 9.4 | 41.9 | 1.0 | 10.3 |
| LP_2_total | 16.0 | 57.0 | 16.6 | 8.1 | 4.5 | 4.8 | 29.5 | 1.0 | 8.4 |
| FP_1_total | 11.6 | 25.1 | 10.3 | 3.8 | 2.2 | 2.7 | 10.4 | 0.6 | 5.0 |
| FP_2_total | 48.7 | 75.0 | 45.3 | 10.2 | 5.5 | 3.9 | 64.7 | 1.0 | 16.2 |
| PostM_1_total | 11.6 | 34.0 | 8.4 | 4.3 | 2.2 | 1.4 | 4.3 | 0.4 | 2.1 |
| PostM_2_total | 10.4 | 21.8 | 9.2 | 3.3 | 1.5 | 1.2 | 8.5 | 0.2 | 2.9 |
| LP_1_free | | 17.2 | | | | | 19.8 | | 4.5 |
| LP_2_free | | 15.7 | | | | | 11.1 | | 3.5 |
| FP_1_free | | 9.3 | | | | | 7.3 | | 2.3 |
| FP_2_free | | 23.9 | | | | | 27.3 | | 7.6 |
| PostM_1_free | | 7.5 | | | | | 2.6 | | 1.1 |
| PostM_2_free | | 8.3 | | | | | 4.9 | | |

| Sample Name | E1 | 4ME2 | E2 | 2OHE1 | 2OHE2 | 4OHE1 | Total | Sample Name |
|---|---|---|---|---|---|---|---|---|
| LP_1_total | 777.9 | 1.4 | 174.2 | 514.6 | 32.7 | 64.4 | 1721.0 | LP_1_total |
| LP_2_total | 671.9 | 0.9 | 122.0 | 201.6 | 48.0 | 31.8 | 1222.0 | LP_2_total |
| FP_1_total | 192.6 | 0.8 | 31.2 | 75.9 | 15.6 | 14.5 | 402.3 | FP_1_total |
| FP_2_total | 1270.2 | 2.5 | 218.5 | 522.8 | 39.6 | 78.5 | 2402.6 | FP_2_total |
| PostM_1_total | 376.7 | 1.0 | 13.2 | 64.0 | 10.5 | 9.2 | 543.4 | PostM_1_total |
| PostM_2_total | 507.4 | 1.1 | 89.7 | 81.0 | 11.6 | 13.7 | 763.4 | PostM_2_total |
| LP_1_free | 58.1 | | 79.8 | | | | 179.5 | LP_1_free |
| LP_2_free | 49.2 | | 65.5 | | | | 145.0 | LP_2_free |
| FP_1_free | 17.2 | | 19.5 | | | | 55.6 | FP_1_free |
| FP_2_free | 83.3 | | 108.2 | | | | 250.2 | FP_2 free |
| PostM_1_free | 24.6 | | 8.7 | | | | 44.5 | PostM_1_free |
| PostM_2_free | 40.7 | | 21.3 | | | | 75.2 | PostM_2_free |

LP: Luteal Phase;
FP: Follicular Phase;
PostM: Postmenopausal
total: unconjugated + conjugated
free: unconjugated

What is claimed:

1. A method of analyzing the presence of estrogens in a biological sample, comprising:
extracting estrogens from a sample to provide a concentrated sample, the estrogens comprising an estrogen, an estrogen metabolite, or any combination thereof;
reacting estrogens in the concentrated sample in a single derivatization step with a hydroxyl protecting reagent under pH conditions between about 7 and about 11.5 in the presence of a reducing agent, an anti-oxidant, or both, to form estrogen derivatives, the concentrated sample optionally comprising at least one ketolic steroid or metabolite thereof, the estrogen derivatives comprising one or more derivatives of one or more estrogens, one or more derivatives of one or more estrogen metabolites, or any combination thereof;
at least partially purifying the estrogen derivatives; and
analyzing the purified estrogen derivatives by mass spectrometry to ascertain the amount of estrogens in the sample.

2. The method of claim 1 wherein the biological sample includes blood, plasma, serum, saliva, lymph fluid, cellular interstitial fluid, mucus, spinal fluid, tissue, breast nipple aspirate, breast duct lavage, urine, or any combination thereof.

3. The method of claim 1 wherein the estrogens or one or more metabolites thereof are catechol estrogens or one or more metabolites thereof.

4. The method of claim 1 wherein the hydroxyl protecting reagent comprises a compound that forms a silyl derivative, an acyl derivative, a benzoyl derivative, an alkyl derivative, a dansyl derivative, a nitrobenzofuran derivative, or any combination thereof.

5. The method of claim 4 wherein the hydroxyl protecting reagent comprises dabsyl chloride, dansyl chloride, 1-fluoro-2,4-dinitrobenzene, 4-fluoro-3-nitrobenzofurazan, or any combination thereof.

6. The method of claim 1 wherein the extracting step includes hydrolyzing conjugates of estrogens present in the sample.

7. The method of claim 6 wherein the atmospheric pressure ionization mass spectrometry comprises positive ion mode electrospray mass spectrometry.

8. The method of claim 1 wherein purifying the estrogen derivatives includes separating the estrogen derivatives.

9. The method of claim 1 wherein the estrogens are reacted with a hydroxyl protecting reagent prior to purifying.

10. The method of claim 8 wherein the estrogen derivatives are purified and separated using HPLC, reverse phase HPLC, gas chromatography, or any combination thereof.

11. The method of claim 10 wherein the reverse phase HPLC is performed using a gradient solution comprising methanol and aqueous formic acid, and a non-polar stationary phase.

12. The method of claim 11 wherein the non-polar stationary phase includes a C18 stationary phase.

13. The method of claim 12 wherein HPLC is performed with gradient elution from about 70:30 to about 85:15, based on volume, of methanol:aqueous formic acid solution.

14. The method of claim 1 wherein the extraction step is carried out with slow inverse extraction using an organic solvent.

15. The method of claim 14 wherein the organic solvent comprises dichloromethane.

16. The method of claim 1 wherein the anti-oxidant is added to the sample prior to the extraction step.

17. The method of claim 16 wherein the anti-oxidant includes an ascorbic acid, a tocopherol, a carotenoid, beta carotene, butylated hydroxyanisole, butylated hydroxytoluene, propyl gallate, trihydroxybutyrophenone, uric acid, or any combination thereof.

18. The method of claim 17 wherein the anti-oxidant includes L-ascorbic acid.

19. The method of claim 1 wherein the anti-oxidant concentration is in the range of from about 0.01 percent (w/v) to about 1 percent (w/v) based on volume of the concentrated sample.

20. The method of claim 1 wherein the pH is in the range of from about 8.5 to 10.5.

21. The method of claim 1 wherein the estrogens are reacted for less than about 10 minutes prior to purifying the estrogen derivatives.

22. A method of detecting a disease state or condition, elevated risk of a disease state or condition, or absence of a disease state or condition in a mammal, comprising:
obtaining a biologic sample from the mammal;
extracting steroids from the biologic sample to provide a concentrated sample, the steroids comprising a steroid, a steroid metabolite, or any combination thereof;
reacting steroids in the concentrated sample in a single derivatization step with a hydroxyl protecting reagent under pH conditions between about 7 and about 11.5 in the presence of a reducing agent, an anti-oxidant, or both, to form steroid derivatives, the concentrated sample optionally comprising at least one ketolic steroid or metabolite thereof, the steroid derivatives comprising one or more derivatives of one or more steroids, one or more derivatives of one or more steroid metabolites, or any combination thereof;
at least partially purifying the steroid derivatives;
analyzing the purified steroid derivatives by mass spectrometry to provide a steroid metabolite profile of the biologic sample; and
comparing the steroid metabolite profile of the biologic sample to at least one steroid metabolite profile indicative of a disease state or condition or absence of a disease state or condition to ascertain the presence or absence of the disease or condition in the mammal or the likelihood of contracting the disease or having the condition by the mammal.

23. The method of claim 22 wherein the steroid or one or more metabolites thereof is an estrogen or one or more metabolites thereof.

24. The method of claim 22 wherein the disease state or condition comprises breast cancer, ovarian cancer, an endocrine disease, infertility, or any combination thereof.

25. A method of testing a mammal for the presence of illegal steroids comprising:
obtaining a biologic sample from the mammal;
extracting steroids from the biologic sample to provide a concentrated sample, the steroids comprising a steroid, a steroid metabolite, or any combination thereof;
reacting steroids in the concentrated sample in a single derivatization step with a hydroxyl protecting reagent under pH conditions between about 7 and about 11.5 in the presence of a reducing agent, an anti-oxidant, or both, to form steroid derivatives, the concentrated sample optionally comprising at least one ketolic steroid or metabolite thereof, the steroid derivatives comprising one or more derivatives of one or more steroids, one or more derivatives of one or more steroids metabolites, or any combination thereof;
at least partially purifying the steroid derivatives;
analyzing the purified steroid derivatives by mass spectrometry to ascertain the amounts of individual steroids in the sample; and
comparing the amounts of individual steroids in the biologic sample with threshold amounts as determined by applicable federal, state, local, association or league rules.

26. The method of claim 25 wherein the mammal is a human.

27. The method of claim 26 wherein the human is an athlete.

28. The method of claim 27 wherein the athlete is a member of the National Basketball Association (NBA), National Hockey League (NHL), National Football League (NFL), Major League Baseball (MLB), National Collegiate Athletic Association (NCAA), Association of Tennis Players (ATP), World Boxing Organization (WBO), World Boxing Association (WBA), World Boxing Council (WBC), or any combination thereof.

29. The method of claim 25 wherein mammal includes a horse, sheep, goat, cat, dog, pig, guinea pig, monkey, cow, rat, or mouse.

30. The method of claim 25, wherein the threshold amount is the detectable limit of an illegal steroid.

31. The method of claim 25 wherein the biologic sample includes blood, plasma, serum, saliva, lymph fluid, cellular interstitial fluid, mucus, spinal fluid, tissue, breast nipple aspirate, breast duct lavage, urine, or any combination thereof.

32. The method of claim 25 wherein the steroids or one or more metabolites thereof are catechol steroids or one or more metabolites thereof.

33. The method of claim 25 wherein the hydroxyl protecting reagent comprises a compound that forms a silyl derivative, an acyl derivative, a benzoyl derivative, an alkyl derivative, a dansyl derivative, a nitrobenzofuran derivative, or any combination thereof.

34. The method of claim 25 wherein the hydroxyl protecting reagent comprises dabsyl chloride, dansyl chloride, 1-fluoro-2,4-dinitrobenzene, 4-fluoro-3-nitrobenzofurazan, or any combination thereof.

35. The method of claim 25 wherein the extracting step includes hydrolyzing conjugates of steroids present in the biologic sample.

36. The method of claim 35 wherein the atmospheric pressure ionization mass spectrometry comprises positive ion mode electrospray mass spectrometry.

37. The method of claim 25 wherein purifying the steroid derivatives includes HPLC.

38. The method of claim 25 wherein the steroids are reacted with a hydroxyl protecting reagent prior to separation by liquid or gas chromatography.

39. The method of claim 37 wherein HPLC includes reverse phase HPLC.

40. The method of claim 39 wherein the reverse phase HPLC is performed using a gradient solution comprising methanol and aqueous formic acid, and a non-polar stationary phase.

41. The method of claim 40 wherein the non-polar stationary phase includes a C18 stationary phase.

42. The method of claim 40 wherein HPLC is performed with gradient elution from about 70:30 to about 85:15, based on volume, of methanol:aqueous formic acid solution.

43. The method of claim 25 wherein the extraction step is carried out with slow inverse extraction using an organic solvent.

44. The method of claim 25 wherein the anti-oxidant includes an ascorbic acid, a tocopherol, a carotenoid, beta carotene, butylated hydroxyanisole, butylated hydroxytoluene, propyl gallate, trihydroxybutyrophenone, uric acid, or any combination thereof.

45. The method of claim 44 wherein the anti-oxidant includes L-ascorbic acid.

46. The method of claim 25 the pH is in the range of from about 8.5 to 10.5.

47. The method of claim 46 wherein the pH is between about 9.0 and about 9.2.

48. The method of claim 25 wherein the steroids are reacted for less than about 10 minutes prior to purifying the steroid derivatives.

49. The method of claim 25 wherein the anti-oxidant concentration is in the range of from about 0.01 percent (w/v) to about 1 percent (w/v) based on volume of the concentrated sample.

50. The method of claim 25 wherein the extraction step is carried out with slow inverse extraction using an organic solvent.

51. The method of claim 50 wherein the organic solvent comprises dichloromethane.

52. The method of claim 25 wherein the anti-oxidant is added to the biologic sample prior to the extraction step.

53. The method of claim 1, further comprising the step of adding a standard of one or more deuterated steroids to the biologic sample, to the concentrated sample, or any combination thereof.

54. The method of claim 22, further comprising the step of adding a standard of one or more deuterated steroids to the biologic sample, to the concentrated sample, or any combination thereof.

55. The method of claim 25, further comprising the step of adding a standard of one or more deuterated steroids to the biologic sample, to the concentrated sample, or any combination thereof.

56. A kit for use in a method for detecting one or more steroids in a biologic sample by mass spectrometry, the kit comprising in packaged combination:
an antioxidant, reducing agent, or any combination thereof;
a standard of one or more deuterated steroids;
a hydroxyl protecting reagent; and
a derivatization buffer characterized as having a pH in the range of from about 7 to about 11.5.

57. The kit of claim 56, wherein the hydroxyl protecting reagent comprises a compound that forms a silyl derivative, an acyl derivative, a benzoyl derivative, an alkyl derivative, a dansyl derivative, a nitrobenzofuran derivative, or any combination thereof.

58. The kit of claim 57, wherein the hydroxyl protecting reagent comprises dabsyl chloride, dansyl chloride, 1-fluoro-2,4-dinitrobenzene, 4-fluoro-3-nitrobenzofurazan, or any combination thereof.

59. The kit of claim 56, wherein the deuterated standard comprises one or more deuterated estrogens.

60. The kit of claim 56, wherein the deuterated standard comprises a deuterated catechol estrogen.

61. The kit of claim 56, wherein the anti-oxidant includes an ascorbic acid, a tocopherol, a carotenoid, beta carotene, butylated hydroxyanisole, butylated hydroxytoluene, propyl gallate, trihydroxybutyrophenone, uric acid, or any combination thereof.

62. The kit of claim 61, wherein the antioxidant includes L-ascorbic acid.

63. The kit of claim 58, wherein the hydroxyl protecting reagent comprises dansyl chloride.

64. The kit of claim 56, wherein the derivatization buffer includes a sodium bicarbonate buffer having a pH in the range of from about 8.5 to about 11.5.

65. The kit of claim 56, further comprising instructions for reacting the components of said kit with steroids in a biological sample.

66. The kit of claim 56, further comprising a hydrolysis buffer.

67. The kit of claim 59, comprising at least five deuterated standards.

68. The method of claim 22, wherein the biologic sample includes blood, plasma, serum, saliva, lymph fluid, cellular interstitial fluid, mucus, spinal fluid, tissue, breast nipple aspirate, breast duct lavage, urine, or any combination thereof.

69. The method of claim 1, wherein the concentrated sample comprises at least one ketolic estrogen or metabolite thereof.

70. The method of claim 22, wherein the concentrated sample comprises at least one ketolic estrogen or metabolite thereof.

71. The method of claim 25, wherein the concentrated sample comprises at least one ketolic estrogen or metabolite thereof.
